(19) United States
(12) Patent Application Publication
(10) Pub. No.: US 2024/0397941 A1
(43) Pub. Date: Dec. 5, 2024

(54) Methods and Compositions for Growing Plants

(71) Applicant: GreenTech Ventures, Inc., Chantilly, VA (US)

(72) Inventors: Zachary Jovan POGUE, Washington, DC (US); Robin Elizabeth ROSS, Monroe, NC (US)

(21) Appl. No.: 18/688,910

(22) PCT Filed: Sep. 2, 2022
(86) PCT No.: PCT/US2022/042498

§ 371 Date: Mar. 4, 2024

Related U.S. Application Data
(60) Provisional application No. 63/240,337, filed on Sep. 2, 2021.

(51) Int. Cl. A01N 25/32; A01N 65/03; A01P 13/02; C12N 1/12

(52) U.S. Cl. CPC A01N 25/32; A01N 65/03; A01P 13/02; C12N 1/12

(57) ABSTRACT

Disclosed herein are compositions comprising supplements for growing plants. Also disclosed are methods of using the compositions and methods of manufacturing the compositions. The compositions can increase cation uptake in plants.

### FIELD

Disclosed herein are compositions comprising supplements for growing plants. Also disclosed are methods of using the compositions and methods of manufacturing the compositions. The compositions can increase cation uptake by plants.

### BACKGROUND

Supplementing or amending the soil in which crops are grown is common practice in agriculture, leading to an improvement in the health of the soil, increase in the population and diversity of beneficial microorganisms in the soil microbiome, the health of plants, improved root system, enhanced growth, and thus an increased yield and improved fruit quality and shelf life for the grower. The application of fertilizers is well known and understood, with most being based around the key nutrients of nitrogen (N), phosphorous (P), and potassium (K). Such fertilizers are typically known as “NPK” fertilizers and are routine in the art. A burgeoning class of supplements (or soil amendments) based around other factors which are important to plant growth and soil health are becoming increasingly used in commercial agriculture. Several common fertilizers that are available are derived from the biomass of other organisms, such as manure, bracken, seaweed, and peat moss. Leaving the traditional fertilizers to one side, there are other types of soil supplements (or amendments) that are used to improve plant and soil health. These typically work by improving the condition of the soil, improving the microbial ecosystem in the soil, and allowing key nutrients to be better assimilated and absorbed by the plant. For example, soil conditioners alter the texture and porosity of the soil in order to encourage root growth, allow increased aeration and improved drainage of the soil.

Cations, such as iron, are key nutrients for crops. It is absorbed by the roots from the soil in an active process, and plants which don't absorb enough iron have reduced yield, suffer chlorosis with a yellowing of the leaves and an overall reduction in plant health. This is a particular problem in soils with a neutral to alkaline pH (i.e., a pH of >7). In soils with these pH values, the iron tends to be trapped in insoluble complexes. This iron can only be efficiently absorbed if the iron is converted into a more absorbable form (such as Fe²⁺) or otherwise is bound to a siderophore. Siderophores are iron-chelating chaperones which enable the transport of iron across cell membranes.

Certain soils naturally have these high pH conditions, such as calcareous soils. These soils are defined by having the presence of significant amounts of calcium carbonate (CaCO₃) and are widespread across the world, especially in drier regions. These soils are naturally high in pH, and while the potential productivity of such soils is high due to good structural characteristics of the soil, the absorbance of micronutrients such as iron and zinc is inhibited by the high pH of the soil. Low-iron or Iron Deficiency Chlorosis (IDC) prone soils can also be created by the incidental use of chelating agents such as glyphosate. It was known that glyphosate has a chelating effect on many cations present in the soil, including iron. Thus, the use of glyphosate can create a low-iron environment in soil which reduces yield and impacts the growth of plants in soils where glyphosate has been used. Other herbicides may have a similar effect including, but not limited to, glufosinate, paraquat and paraquat diquat.

The issue of limited absorbance of cations, such as iron in low-iron, high pH soils, is an issue without an effective solution. Short term approaches involve applying micronutrients to foliage or other symptomatic treatments, or otherwise acidifying the soil to encourage the conversion of iron into a more absorbable form. Clearly, for plants which grow best in neutral to alkaline soils, any treatment which acidifies the soil is not preferable for encouraging maximal yield. Likewise, on commercial farms, the symptomatic treatment of chlorosis is not an efficient solution to the problem of chlorosis. What is needed is a treatment for such soils that does not alter the pH of the soil but encourages the uptake of iron in plants that grow best in these soils. Additionally, what is needed is a treatment which counteracts the negative effects of herbicide treatment on soil microbial communities to improve iron-uptake in plants.

### SUMMARY OF THE INVENTION

Disclosed herein are methods of increasing cation uptake in a plant growing in soil treated with an herbicide or pesticide, comprising: (a) applying an effective amount of an herbicide or pesticide to the plant and/or soil; and (b) within 10 days after the applying in (a), applying to the plant and/or soil an effective amount of a composition comprising one or more gibberellins, one or more auxins, salicylic acid, and/or one or more jasmonates. In some embodiments, the applying in (a) and (b) occur concurrently or on the same day. In some embodiments, the applying in (b) occurs 1 to 9 days after the applying in (a). In some embodiments, the applying in (b) occurs 1 to 5 days after the applying in (a). In other embodiments, the methods can further comprise applying the composition every 1 to 60 days after the applying in (b).

Also disclosed are methods of enhancing cation-uptake or iron-uptake in plants growing in low-cation or low-iron soil, comprising applying an effective amount of a composition comprising one or more gibberellins, one or more auxins, salicylic acid, and/or one or more jasmonates to one or more seeds, the soil, growing media, and/or a cultivated area. In some embodiments, an herbicide or pesticide is applied to the one or more seeds, the low-cation, low-iron or Iron Deficient Chlorosis (IDC)-prone soil, growing media, and/or a cultivated area concurrently, on the same day, or within 10 days before the composition is applied. In some embodiments, the effective amount is applied to the low-cation, low-iron or Iron Deficient Chlorosis (IDC)-prone soil, growing media, and/or the cultivated area, and wherein the effective amount is 1 to 5 liters per acre.

Further disclosed herein are methods of producing a composition that increases cation uptake in a plant growing in soil, comprising providing an algae culture and air to an algae culture broth composition producing system, the system comprising:
a sterilizer;
an automatic carbon dioxide supply device to promote photosynthesis;
an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and
a high-efficiency harvesting device using hollow fiber membranes; and
harvesting the composition.

The composition can be harvested by separating the composition from biomass of the algae culture using a harvesting device, optionally dehydrating the composition for storage and transport, and optionally diluting it prior to use.

Also disclosed herein are mixtures comprising an herbicide or pesticide and a composition comprising one or more gibberellins, one or more auxins, salicylic acid, and/or one or more jasmonates. In some embodiments, prior to an optional dilution, the composition comprises: the one or more gibberellins are at a concentration of 0.01 to 1 ng/ml; the one or more auxins are at a concentration of 0.001 to 1 ng/ml; the salicylic acid is at a concentration of 0.1 to 1 ng/ml; and/or the one or more jasmonates are at a concentration of 0.001 to 1 ng/ml.

Also disclosed herein are the use of a composition to enhance the cation uptake or iron-uptake in plants growing in low-cation, low-iron or Iron Deficient Chlorosis (IDC)-prone soils, the composition comprising: one or more gibberellins; one or more auxins; salicylic acid; and/or one or more jasmonates.

In some embodiments, prior to an optional dilution of the applying in (b), the composition comprises the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01-1 ng/ml, the one or more auxins are at a concentration of 0.001 to 0.5 ng/ml, the salicylic acid is at a concentration of 0.5-1 ng/ml, and/or the one or more jasmonates are at a concentration of 0.001 to 0.1 ng/ml; or the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml.

The one or more gibberellins can comprise GA1, GA3, GA4, GA5, GA6, GA7, or combinations thereof. The one or more auxins can comprise Me-IAA, IAA-ALA, IAA-ASP, IBA, or combinations thereof. The one or more jasmonates can comprise jasmonic acid.

The composition can be derived from algae, macroalgae, and/or microalgae. The microalgae can comprise Chlorella, Spirulina, Nannochloropsis, and/or Scenedesmus or combinations thereof.

The composition further comprises cultured and enhanced sterilized water or aqueous media, optionally at 80% to 99.5% w/w.

The composition can comprise biomass, ethylene, abscisic acid, brassinolides, or combinations thereof. In other embodiments, the composition does not comprise biomass, ethylene, abscisic acid, brassinolides, or combinations thereof.

The plant can be from any of the following families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythruceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.

The methods can further comprise diluting the composition 50:1 and 300:1 and performing one or more additional applications of the effective amount of the composition. The methods can further comprise additionally applying the composition every 1 to 60 days. In some embodiments, the effective amount is applied to the low-cation, low-iron or Iron Deficient Chlorosis (IDC)-prone soil, growing media, and/or the cultivated area, and wherein the applying comprises injection, in-furrow, drip irrigation, center-pivot, surface broadcast, broadcast incorporated, band application, fertigation, chemigation, foliar application, sidedress, topdress, seed placement, seed treatment, or combinations thereof.

### BRIEF DESCRIPTION OF THE FIGURES

FIGS. 1A-1D show the results of application of the composition to strawberry plants, including the fold change % in the relative abundance of Pseudomonas due to the application of the composition per time and location (FIG. 1A), the fold change % in the relative abundance of Bacillus due to the application of the composition per time and location (FIG. 1B), the fold change % in the relative abundance of Achromobacter due to the application of the composition per time and location (FIG. 1C), and the fold change % in the relative abundance of Cytobacillus due to the application of the composition per time and location (FIG. 1D).

FIGS. 2A-2D show representative examples of the roots and foliage of strawberry plants taken from untreated control lots (FIGS. 2A and 2C) and those treated with the composition (FIGS. 2B and 2D).

FIG. 3 shows soybean pods harvested from untreated control lots and lots treated with the composition disclosed herein.

FIG. 4 shows soybean pods harvested from untreated control lots and lots treated with the composition disclosed herein.

FIG. 5 shows soybean foliage from untreated control lots and lots treated with the composition disclosed herein.

FIG. 6 shows soybean foliage from untreated control lots and lots treated with the composition disclosed herein.

FIG. 7 shows the root system and root nodules of a soybean plant representative of those harvested from an untreated control lot.

FIG. 8 shows the root system and root nodules of a soybean plant representative of those harvested from a lot treated with the composition disclosed herein.

FIG. 9 shows representative examples of soybean plants and their root systems from untreated control lots and those harvested from lots treated with the composition disclosed herein.

FIG. 10 shows an untreated control lot in which soybean plants are growing and a plot treated with the composition disclosed herein in which soybean plants are growing.

FIG. 11 shows a process flow diagram for producing a composition disclosed herein.

FIG. 12 shows an overview of the process for producing a composition disclosed herein.

### DETAILED DESCRIPTION OF THE INVENTION

It has been determined that compositions comprising one or more gibberellins and/or one or more auxins and/or salicylic acid and/or one or more jasmonates can be used to vastly improve cation update by plants, such as iron-uptake in plants growing in low-iron or iron-deficient chlorosis (IDC)-prone soils. Cations, such as iron, are key nutrients for plant growth and health, and this aspect of plant biology is well known (see Malo D., 2013). A “low-iron soil” or “IDC-prone soil” can be taken to mean any soil in which the ability of plants to uptake iron from the soil is sub-optimal. In these soils, just because iron is present, even in large quantities, it may be not bioavailable to the plant, requiring assistance to be converted into a soluble form. Such soils can have an abundance of iron which is trapped in forms unsuitable for efficient uptake by plants. For example, high pH soils (pH >7) can contain an abundance of ferric iron (Fe³⁺) and a lack of the more readily absorbable ferrous iron (Fe²⁺).

Other low-iron or Iron Deficiency Chlorosis (IDC) prone soils can be those which have been treated with a chelating agent, or an agent known to have chelation properties. Of particular note is the ability of glyphosate for chelating iron in soil. Glyphosate use can lead to a low-iron soil condition (Mertens et al., 2018). Thus, despite glyphosate being used as an herbicide for growing glyphosate resistant crops, it can still have an impact on the availability of bioavailable cations, such as iron, and thus impact the health of crops grown in treated soils, as has been shown in glyphosate resistant soybeans, where the root nodule formation was impacted by glyphosate use despite the resistance of the plants (see e.g., Zobiole et al., 2011). This effect is not limited to glyphosate, other soils where the application of nutrient binding chemicals, including, but not limited to, glufosinate, results in chelated cation nutrients, including zinc, iron, manganese, and aluminum, and changes in relative abundance of fungi and bacterial taxa.

As a further example of a low-iron or Iron Deficiency Chlorosis (IDC) prone soil, calcareous soils are known to be a contributing factor to iron chlorosis in plants (R. H. Loeppert, 2008). Calcareous soils can be defined as any soil having a significant amount of Calcium carbonate, with one study defining a calcareous soil as a soil having 8% and 10% calcium carbonate (Hilal et al., 1973). These soils have a buffering capacity which maintains the high pH of the soil and reduces the ability of plants to convert ferric iron into ferrous iron. Surprisingly, applicants have found that treatment with the above-mentioned composition improves iron uptake in plants grown in these soils.

Techniques for determining whether a soil is classed as a “low-iron” or “IDC-prone” soil are known. One such technique is the Conventional DTPA-Fe soil test (Lindsay & Norvell, 1978). This test allows the extraction and measurement of soil micronutrient amounts, including iron. Thresholds for defining iron availability are different from plant to plant (outside of the extreme ranges of low and high). As an example, the iron thresholds are as follows:
Low—0-2.5 ppm,
Medium—2.6-4.5 ppm,
High—4.6-10 high,
Very High—10+

These threshold values indicate the level of iron in the soil, with soils having low and medium iron levels considered to be IDC-prone. The optimal values are derivable for plants using routine empirical scientific methods (see e.g., Sakal et al., 1984).

Another available soil health test is the Haney Soil Health Tool (Haney et al., 2018). As before, the iron thresholds for the risk of IDC are readily derivable by the skilled person, but the following are provided as an example:
Low—0-7.5 ppm,
Medium—7.6-13.5 ppm,
High—13.8-30,
Very High—30+

As with the DTPA-Fe test, the threshold values indicate the level of iron in the soil, with soils having low and medium iron levels considered to be IDC-prone. The optimal values for plants are derivable for plants using routine empirical scientific methods.

The above ranges are, in some embodiments, used for defining the soil in the case of soybeans and also other plants or crops that require similar ranges, or their equivalents. Equivalent ranges for non-soya plants or non-soya crops are also apparent to the skilled person. It should be noted that high and very high levels of iron are not thought to negatively impact plant health, unlike e.g., Zinc.

As used herein, the terms “increasing,” “enhancing,” and “improving” can be used interchangeably and can be compared to a control without the compositions or performance of the methods disclosed herein.

As used herein, the term “uptake” means absorption, such as by a plant. As used herein, a “plant” includes all its parts, including but not limited to leaves, stems, fruits, flowers, roots, and root system.

Herbicides and pesticides are well known in the art and as used herein, can include but are not limited to anionic, negatively charged, or cation-chelating herbicides and pesticides, such as but not limited to Glyphosate (Roundup®), Glufosinate (Liberty or Basta), and Atrazine.

As used herein, a plant” growing in soil treated with an herbicide or pesticide” means that the plant, parts of the plant, and/or soil in which the plant is growing has been treated with the herbicide and/or pesticide.

As used herein, unless specified, “soil” includes any soil suitable for growing plants, including soils that are particularly suitable for growing a particular plant, low-cation, soils, where cation uptake is inhibited, challenged, or suboptimally mobilized/solubilized/utilized, such as, but not limited to, iron-deficient or iron deficiency chlorosis (IDC) prone, due to the introduction of certain soil inputs, such as herbicides or pesticides, or due to high pH or similar alkaline soil effects.” Thus, in some embodiments, “low-cation” or “low-iron” soil means a condition where the cation or iron quantity is low or suboptimal for growing the plant of interest or when there is sufficient cation or iron but the cation or iron is inhibited, challenged, or suboptimally mobilized/solubilized/utilized compared to a condition wherein soil inputs are not present.

As used herein, cations include but not limited to iron, potassium, phosphorous, zinc, manganese, aluminum, magnesium, and/or combinations thereof. It will be understood that a “low-cation soil,” as with a “low-iron soil,” does not necessarily mean that there is a low concentration of these cations in the soil, but instead it can mean that the cations in such soils are not readily available for uptake by the plant. The soil can be tested using the same protocols as outlined above for iron (for example, the Haney Soil Health Tool). The compositions and methods disclosed herein improve the uptake of cations in plants, and as such has beneficial effects on the health of the plants.

Disclosed herein are methods of increasing cation uptake in a plant growing in soil treated with an herbicide or pesticide, comprising: (a) applying an effective amount of an herbicide or pesticide to the plant and/or soil; and (b) within 10 days after the applying in (a), applying to the plant and/or soil an effective amount of a composition comprising one or more gibberellins, one or more auxins, salicylic acid, and/or one or more jasmonates. In some embodiments, the applying in (a) and (b) occur concurrently or on the same day. In some embodiments, the applying in (b) occurs 1 to 9 days after the applying in (a). In some embodiments, the applying in (b) occurs 1 to 5 days after the applying in (a). In some embodiments, the applying in (b) occurs 1, 2, 3, 4, 5, 6, 7, 8, 9, or 10 days after the applying in (a). As used herein, the term “within” 10 days includes the 10^{th} day, e.g., if an herbicide or pesticide is applied on Day 1, and a composition disclosed herein is applied “within” 10 days, it would include applying up to Day 11. The methods disclosed herein refers to each time an herbicide or pesticide is applied, such that each time a plant is applied with an herbicide or pesticide, the methods disclosed herein can be utilized. For example, if a plant is applied with an herbicide or pesticide multiple times during its lifecycle, the methods disclosed herein can be utilized after each application of the herbicide or pesticide.

In other embodiments, the methods can further comprise applying the composition every 1 to 60 days after the applying in (b). In some embodiments, the methods can further comprise applying the composition once every 1 to 30 days, once per month, once every 1 to 14 days, once every 7 days, or once every 5 days, or once every number of days therein.

Also disclosed are methods of enhancing cation-uptake or iron-uptake in plants growing in low-cation or low-iron soil, comprising applying an effective amount of a composition comprising one or more gibberellins, one or more auxins, salicylic acid, and/or one or more jasmonates to one or more seeds, the low-cation, low-iron or Iron Deficient Chlorosis (IDC)-prone soil, growing media, and/or a cultivated area. In some embodiments, an herbicide or pesticide is applied to the one or more seeds, the low-cation, low-iron or Iron Deficient Chlorosis (IDC)-prone soil, growing media, and/or a cultivated area concurrently, on the same day, or within 10 days before the composition is applied. In some embodiments, the effective amount is applied to the low-cation, low-iron or Iron Deficient Chlorosis (IDC)-prone soil, growing media, and/or the cultivated area, and wherein the effective amount is 1 to 5 liters per acre.

Further disclosed herein are methods of producing a composition that increases cation uptake in a plant growing in soil, comprising providing an algae culture and air to an algae culture broth composition producing system, the system comprising:
a sterilizer;
an automatic carbon dioxide supply device to promote photosynthesis;
an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and
a high-efficiency harvesting device using hollow fiber membranes; and
harvesting the composition.

The composition can be harvested by separating the composition from biomass of the algae culture using a harvesting device, optionally dehydrating the composition for storage and transport, and optionally diluting it prior to use.

Further disclosed herein are methods of producing compositions that increase cation uptake in a plant growing in soil as described herein and further comprising applying the composition to a plant by the methods described herein.

Also disclosed herein are mixtures comprising an herbicide or pesticide and a composition comprising one or more gibberellins, one or more auxins, salicylic acid, and/or one or more jasmonates. In some embodiments, prior to an optional dilution, the composition comprises: the one or more gibberellins are at a concentration of 0.01 to 1 ng/ml; the one or more auxins are at a concentration of 0.001 to 1 ng/ml; the salicylic acid is at a concentration of 0.1 to 1 ng/ml; and/or the one or more jasmonates are at a concentration of 0.001 to 1 ng/ml.

Also disclosed herein are the use of a composition to enhance the cation-uptake or iron-uptake in plants growing in low-cation, low-iron or Iron Deficient Chlorosis (IDC)-prone soils, the composition comprising: one or more gibberellins; one or more auxins; salicylic acid; and/or one or more jasmonates.

In some embodiments, prior to an optional dilution of the applying in (b), the composition comprises the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01-1 ng/ml, the one or more auxins are at a concentration of 0.001 to 0.5 ng/ml, the salicylic acid is at a concentration of 0.5-1 ng/ml, and/or the one or more jasmonates are at a concentration of 0.001 to 0.1 ng/ml; or the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml. In some embodiments, the mixtures comprising an herbicide and/or pesticide and a composition disclosed herein can be diluted before applying to the plants.

Gibberellins are a large family of plant hormones involved in various aspects of plant growth. There are approximately 126 known gibberellins which have been identified in plants, fungi, and bacteria. Of these, there is a known subset of active gibberellins which are thought to be key in regulating biological processes in plants. In some embodiments, the gibberellins comprise GA1, GA3, GA4, GA5, GA6, GA7, GA8, GA9, GA13, GA14, GA15, GA19, GA20, GA24, GA29, GA44, GA51, and/or GA53, or any combination thereof. In some embodiments, the gibberellins comprise GA1, GA3, GA4, GA6, GA7, GA8, GA9, GA13, GA14, GA15, GA19, GA20, GA24, GA29, and/or GA44. In some embodiments, a composition comprising one or more gibberellins comprising any one of GA1, GA3, GA4, GA5, GA6 and/or GA7, or any combination thereof can produce an enhanced uptake of cations, such as iron, in plants grown in a low-cation, low-iron or Iron Deficiency Chlorosis (IDC) prone soil. In some embodiments, the composition comprises GA1, GA3, GA4, GA6, and/or GA7 or any combination thereof. Surprisingly, applicants have found that a low concentration of gibberellins maintains this effect. While higher concentrations of gibberellins can be beneficial, it has been surprisingly found that even very low concentrations of gibberellins produce the advantageous properties of the compositions disclosed herein. In some embodiments, the gibberellins are in the composition at a concentration of 0.01 to 1 ng/ml, 0.05 to 1 ng/ml, or 0.01 to 0.8 ng/ml. In some embodiments, these concentrations are prior to a dilution into water or aqueous media before use. The gibberellins are known to stimulate material mobilization during germination, stimulate pollen tube growth and promote elongation of stems.

Auxins are a different family of plant hormones which control the growth of plants by promoting mitosis and elongation of plant cells. In some embodiments, the auxins in the present composition comprise Me-IAA, IAA, IBA, IAA-ASP, IAA-GLU, and/or IAA-ALA, or any combination thereof. In some embodiments, the auxins in the present composition comprise M--IAA, IBA, IAA-ASP, and IAA-ALA. While higher concentrations of auxins can be beneficial, it has been surprisingly found that even very low concentrations of auxins produce the advantageous properties of the compositions disclosed herein. In some embodiments, the auxins are in the composition at a concentration of 0.001 to 1 ng/ml, 0.001 to 0.5 ng/ml, or 0.001 to 0.4 ng/ml. In some embodiments, these concentrations are prior to a dilution into water or aqueous media before use.

Salicylic acid is known to regulate genes involved in plant defense mechanisms, enhancing germination, flowering, and ripening. While higher concentrations of salicylic acid can be beneficial, it has been surprisingly found that even very low concentrations of salicylic acid produce the advantageous properties of the compositions disclosed herein. In some embodiments, salicylic acid is present in the composition as a concentration of 0.1 to 1 ng/ml, 0.5 to 1 ng/ml, 0.2 to 0.8 ng/ml, or 0.5 to 0.75 ng/ml. In some embodiments, these concentrations are prior to a dilution into water or aqueous media before use.

Jasmonates are known to play a role in germination and the response to environmental stresses. In some embodiments, the jasmonates of the composition comprise jasmonic acid, 12-oxophytodienoic acid (OPDA), and/or jasmonoyl-isoleucine (JA-Ile) or any combination thereof. While higher concentrations of jasmonates can be beneficial, it has been surprisingly found that even very low concentrations of jasmonates produce the advantageous properties of the compositions disclosed herein. In some embodiments, the jasmonates of the composition comprise jasmonic acid. In some embodiments, the concentration of jasmonates is 0.001 to 1 ng/ml, 0.001 to 0.1 ng/ml, or 0.001 to 0.004 ng/ml. In some embodiments, these concentrations are prior to a dilution into water or aqueous media before use.

Such compositions as those described above have many advantages for plants grown in soils, including but not limited to low-cation, low-iron or Iron Deficiency Chlorosis (IDC) prone soils. They improve cation availability and transport, improve the production of siderophores and thereby elicit siderophore effects (including, but not limited to, scavenging and transport of iron for microbes and crops/plants, especially in soils with high pH and crops/plants containing limited iron micronutrient), improve microbial and fungal biodiversity, and improve utilization efficiency of phytochemicals (including primary and secondary metabolites) by soil microbiome and crops/plants.

Any of the abovementioned compositions can be derived from algae. It is appreciated that where the above composition is not derived from algae, it could be reconstituted by dilution of the relevant constituents under controlled conditions into an appropriate diluent (e.g., water or aqueous media). In some embodiments, the algae are macroalgae, such as seaweed or seaweed extract. In some embodiments, the algae are microalgae, such as those from the genus Chlorella, Spirulina, Nannochloropsis and/or Scenedesmus.

Such algal strains can include but are not limited to:
Chlorella vulgaris Beijerinck 9765™ (ATCC 9765 (Strain designation L756a))
Chlorella sp. 50258™ (ATCC 50258 (Strain designation NC64A)
Chlorella vulgaris Beijerinck 30821™ (ATCC 30281)
Chlorella vulgaris var. viridis Chodat 16487™ (ATCC 16487 (Strain designation UTEX 30))
Scenedesmus obliquus (Turpin) Kutzing 11457 [UTEX 72]
Scenedesmus obliquus (Turpin) Kutzing 11477 Fager D-3 PB6A
Scenedesmus obliquus (Turpin) Kutzing 30580 UTEX 72 [CCAP-276/3a]
Scenedesmus obliquus (Turpin) Kutzing 30431 UTEX 79 [CCAP-276/1b]
Scenedesmus obliquus (Turpin) Kutzing 30430 UTEX 78 [CCAP-276/3b]
Scenedesmus obliquus (Turpin) Kutzing 30434 UTEX 83 [CCAP-276/1a]
Scenedesmus obliquus (Turpin) Kutzing 14477 UTEX 74
Spirulina major (UTEX LB 552)
Spirulina maxima (UTEX LB 2342)
Spirulina platensis (ATCC 53844 (Strain designation M-185))
Spirulina platensis subsp. siamese (ATCC 53843)
Spirulina platensis (UTEX LB 2340)
Spirulina platensis (UTEX LB 1926)
Spirulina platensis (UTEX LB 1928)
Spirulina sp. (UTEX LB 2179)
Spirulina sp. (UTEX SP49)
Spirulina sp. (UTEX LB 770)
Spirulina subsalsa (UTEX LB 1954)
Spirulina subsalsa f. versicolor (UTEX LB 1318)
Nannochloropsis sp. (KMMCC-290)

In some embodiments, the compositions disclosed herein can further comprise cultured and enhanced sterilized water. In this context, cultured means water in which a broth of biologically active algae has interacted and decayed, expressing secretomes into the water prior to the removal of the algal biomass. Enhanced sterilized water means that the water has received one or more sterilization treatments (e.g., micro air bubble or ozone treatment), which results in the optimized distribution of nutrients and injected CO₂. Furthermore, such water can be enhanced with microbubbles injected therein which increases the mixing and agitation of the various constituents of the broth, which in turns increases the productivity of the culture therein.

In some embodiments, the compositions disclosed herein comprise biomass, ethylene, abscisic acid, brassinolides, or combinations thereof. In some embodiments, the compositions disclosed herein does not comprise biomass, ethylene, abscisic acid, brassinolides, or combinations thereof. The inclusion of biomass, including but not limited to cells, cell parts and cellular debris other than secretomes and/or exudates, in the composition is thought to cause acidification of the soils to which it is applied, which will alter the pH of the soil and can be detrimental to plants which grow best at certain pH ranges. As such, in some embodiments, the absence of biomass confers the benefit that the pH level of the soil is not disturbed upon application of the composition lacking said biomass. Ethylene is known to cause the ripening of fruits, and while this is beneficial in some contexts, in some embodiments, ethylene does not form part of the composition. Abscisic acid is known to promote abscission and dormancy in seeds and buds. In some embodiments, abscisic acid is not form part of the present compositions. In some embodiments, the composition does not comprise brassinolides. Brassinolides are known to be involved in various plant processes such as elongation, flowering, and cell division.

By way of non-limiting examples, plants that can be benefitted by growing using the compositions and methods disclosed herein include any of the following families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.

In some embodiments, the plants growing in low-cation, low-iron or Iron Deficiency Chlorosis (IDC) prone soils are strawberries, blueberries, watermelon, tomatoes, almonds, soybeans, corn (also referred to as maize), wheat, table grapes, wine grapes, lettuce, potatoes, sugar beets, sugarcane, turf, cannabis, hemp, avocado, carrot, chili peppers, rice and/or spinach.

In further aspects, disclosed herein are methods of enhancing cation-uptake or iron-uptake in plants growing in low-cation, low-iron or Iron Deficiency Chlorosis (IDC) prone soil comprising providing a composition disclosed herein, or any embodiments thereof disclosed herein, and applying an effective amount of the composition to one or more seeds, low-cation, low-iron or Iron Deficiency Chlorosis (IDC) prone soil, growing media, and/or a cultivated area, or any combination thereof. The growing media can comprise bark, clay, coir pith, green compost, grow foam, black peat, white peat, perlite, rice hulls and/or wood fibers. A cultivated area is defined as an area that is cropped every season for seasonal crops (e.g., rice, maize, wheat, soybeans, and the like). In the case of longer season crops like sugarcane or perennial crops like fruit trees, the cultivated area is cropped every year. In some embodiments, the cultivated area can be tilled.

While higher concentrations of each of the constituents of the composition are thought to produce the benefits associated with the compositions of the present invention, it has been surprisingly found that even very low concentrations of each constituent maintain this beneficial effect. In some embodiments, the composition comprises the composition comprises the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01-1 ng/ml, the one or more auxins are at a concentration of 0.001 to 0.5 ng/ml, the salicylic acid is at a concentration of 0.5-1 ng/ml, and/or the one or more jasmonates are at a concentration of 0.001 to 0.1 ng/ml; the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentration or ranges of concentrations therein, and the method further comprises diluting the composition to 25:1 to 150:1, 50:1 to 150:1, or 100:1 ratio of water (or aqueous media) to composition (water:composition) prior to applying the effective amount. For example, the above dilution range would be 25 to 150 parts water to 1 part of the composition disclosed herein. In some embodiments, the diluent is water or other aqueous media.

In some embodiments, the composition is applied to low-cation, low-iron or Iron Deficiency Chlorosis (IDC) prone soil, growing medium and/or a cultivated area and the effective amount is 0.5 to 1 gallon (1.89 to 3.79 liters, respectively) per acre per week. This is thought to be a particularly advantageous range of volumes at which the composition enhances the iron-uptake in the crop, while minimizing water use.

In some embodiments, the compositions disclosed herein is diluted to 50:1 to 300:1 or 250:1 to 300:1, water:composition ratio, in water prior to performing one or more additional applications of the effective amount of the composition. In some embodiments the diluent is water or other aqueous media.

Such additional applications can be performed every 1 to 60 days, 1 to 30 days, 1 to 14 days, 1 to 7 days, or any days or ranges of days therein. This is thought to be an advantageous schedule for repeated applications of the composition to improve cation-uptake or iron-uptake from low-cation, low-iron or Iron Deficiency Chlorosis (IDC) prone soils.

In some embodiments, the composition is applied to low-cation, low-iron or Iron Deficiency Chlorosis (IDC) prone soil, growing medium, and/or the cultivated area the applying comprises one or more of injection, in-furrow, drip irrigation, center-pivot, surface broadcast, broadcast incorporated, band application, fertigation, chemigation, foliar application, sidedress, topdress, or seed placement and/or seed treatment, or any combination thereof.

In this context, injection is a method of applying the composition through an irrigation system using water to distribute the composition.

In-furrow is a method of spraying over the seeds in the open seed furrow during planting and can be combined with banded application.

Furrow irrigation is a method of laying out the water channels in such a way where gravity limits the amount of water (or composition) to a suitable amount for the growth of the crop, usually made by the planned placement of ridges and furrows.

Drip irrigation is a type of micro-irrigation system that saves water and nutrients by allowing the composition to drip slowly proximal to the roots of plants either from above the soil surface or buried below the surface. This method minimizes evaporation, and the composition is distributed through a network of valves, pipes, tubing, and emitters.

Surface broadcast is the application of the composition by gravity flow to the surface of the field and can also be considered a type of “flood irrigation.”

Broadcast, which can be incorporated or unincorporated, refers to distributing the composition in a uniform manner on the surface of the soil and/or growing medium. This can be a foliar application using any standard broadcast applicator. When applied in this way after planting, this is often referred to as “topdress” application, but when it is incorporated into the soil, it is referred to as broadcast incorporated. This mode of application places a major portion of the composition into the soil zone such that moisture is not available for weeds, but is available for the crop.

Center-pivot is a technique in which water (e.g., containing the composition disclosed herein) is dispersed through an apparatus having a long and segmented arm revolving around a deep central well. Application in this manner can cover a circular area from a quarter of a mile to a mile in diameter. This form of application can be used in the presently claimed method.

Band application, also referred to as “starter application,” is a form of applying the composition to a crop by placing a band proximal to the seed during planting. It can be applied above, below, on one side and/or on both sides of the seed and/or seedlings. The band is close enough to efficiently supply the young plants with the composition, but not so close as to damage the developing roots. Typically, the band is placed at least 2 inches from the seed and/or seedling. When performed after planting, this is known as “sidedress.”

Fertigation is the injection of fertilizers into an irrigation system. It is closely related to chemigation, where chemicals are injected using an irrigation system. Either term can be used with the present method of applying the composition.

Sidedress is a form of adding the composition by mixing it into the soil along each row of crops, which commonly uses a post directed/angled spray nozzle applicator and sometimes in banded fashion.

Topdress involves the uniform distribution of material upon the soil surface and is similar to banded application.

Seed treatment means that seeds are treated with the compositions disclosed herein and/or in a tank mix with other seed treatable products prior to planting. Seed treatment using the composition is thought to be particularly advantageous as it can improve germination and avoids unnecessary wastage of the composition compared to in-soil application where composition can be wasted on weeds, crop residue and soil in which no root systems will develop.

Seed placement means that the composition is placed into the ground proximal to the seed, and thus closer to the root system once it develops.

Further aspects provides methods of enhancing iron-uptake in plants grown in low-cation, low-iron or Iron Deficiency Chlorosis (IDC) prone soils, comprising providing an algae culture and air to an algae culture broth composition producing system, the system comprising: a sterilizer, an automatic carbon dioxide supply device to promote photosynthesis, an at least partially sealed or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration, and a high-efficiency harvesting device using hollow fiber membranes, and harvesting the composition by separating the composition from biomass of the algae culture using the harvesting device, optionally comprising dehydrating the composition for storage and transport and diluting it prior to use, wherein the method comprises applying an effective amount of the composition to one or more seeds, the low-cation, low-iron or Iron Deficiency Chlorosis (IDC) prone soils, growing media, and/or a cultivated area.

Such a system could be implemented in a variety of ways using any number of devices. For example, see WO2018009575A1, which is incorporated herein by reference in its entirety, which demonstrates one example system for achieving the compositions disclosed herein. In some embodiments, the sterilizer is used to sterilize the diluent (e.g., water or aqueous media), and comprises any one or more of the following devices or techniques for doing so, or any combination thereof: a fine filtration device, an ultra-violet light emitting device, a reverse osmosis device, a nano/micro-bubble device, an oxygen delivery device, and/or an ozone delivery device. The use of bubbles through the diluent causes solids to rise to the surface for removal, and the introduction of ozone creates hydroxyl radicals (OH) which sterilize the diluent efficiently.

The at least partially sealed or fully sealed vertical photobioreactor allows for optimal light penetration into the culture which increases productivity thereof. The harvested algae is passed through hollow-fiber membranes to separate the biomass (algal cells and debris) from the compositions disclosed herein.

In any of the aspects or embodiments disclosed herein, the composition can be derived from a microalgae culture comprising Chlorella, Spirulina, Nannochloropsis and/or Scenedesmus. It will be appreciated that any of the embodiments relating to the composition and/or the method of application relevant to the first two aspects are equally applicable to this third aspect.

In further aspects, disclosed herein are methods of enhancing the cation-uptake in plants growing in cation soil, comprising providing an algae culture and air to an algae culture broth composition producing system, the system comprising: a sterilizer; an automatic carbon dioxide supply device to promote photosynthesis; an at least partially sealed or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and a high-efficiency harvesting device using hollow fiber membranes; and harvesting the composition by separating the composition from biomass of the algae culture using the harvesting device, optionally substantially dehydrating the composition for storage and transport, and diluting it prior to use, and applying an effective amount of the composition to one or more seeds, the low-cation soil, growing media, and/or a cultivated area.

Any of the aspects or embodiments disclosed herein can be combined with any of the embodiments outlined relating to aspects of the composition and methods related to low-cation, low-iron or IDC-prone soil. As used herein, cations include but not limited to iron, potassium, phosphorous, zinc, manganese, aluminum, magnesium, and/or combinations thereof. It will be understood that a “low-cation soil,” as with a “low-iron soil,” does not necessarily mean that there is a low concentration of these cations in the soil, but instead it can mean that the cations in such soils are not readily available for uptake by the plant. The soil can be tested using the same protocols as outlined above for iron (for example, the Haney Soil Health Tool). The compositions and methods disclosed herein improve the uptake of cations in plants, and as such has beneficial effects on the health of the plants. In some embodiments, the composition improves the uptake of nutrients in the plants grown in the low-cation, low-iron, and/or IDC-prone soil in which the plants are grown. In some embodiments, the composition improves the soil health of the low-cation, low-iron, and/or IDC-prone soil in which the plants are grown. The compositions and methods disclosed herein can enhance soil native plant growth promoters that have the potential to promote P, K and iron uptake and phytohormone production (all together or individually, in difference balance depending of the soil microbiome status before adding the product). The compositions and methods disclosed herein can improve both nutrient and cation cycling, mobilization, regulation, and uptake, especially phosphorus, potassium and iron, as an example. The compositions and methods disclosed herein can also promote the growth of plant growth promoters (PGP) and plant growth promoting rhizobacteria (PGPR) on the field, which stimulate phytohormone production and stress adaptation.

Disclosed herein are uses of a composition to enhance the iron-uptake in plants growing in low-iron or Iron Deficiency Chlorosis (IDC) prone soils, the composition comprising one or more gibberellins and/or one or more auxins and/or salicylic acid and/or one or more jasmonates.

In some embodiments, the composition comprises one or more gibberellins, one or more auxins salicylic acid, and one or more jasmonates.

In some embodiments, prior to an optional dilution at the point of application to the soil, the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01 to 1 ng/ml, the one or more auxins are at a concentration of 0.001 to 0.5 ng/ml, the salicylic acid is at a concentration of 0.5 to 1 ng/ml, and/or the one or more jasmonates are at a concentration of 0.001 to 0.1 ng/ml; the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentrations or ranges of concentrations therein.

In further embodiments, the composition is derived from algae, optionally macroalgae and/or microalgae. In some embodiments, the microalgae comprise Chlorella, Spirulina, Nannochloropsis, and/or Scenedesmus, or any combination thereof.

In further embodiments, the one or more gibberellins comprise any one of GA1, GA3, GA4, GA5, GA6, and/or GA7 or any combination thereof.

In further embodiments, the one or more auxins comprise any one of Me-IAA, IAA-ALA, IAA-ASP, IBA, or any combination thereof.

In further embodiments, the one or more jasmonates comprise jasmonic acid.

In further embodiments, the composition further comprises cultured and enhanced sterilized water, optionally at 80% to 99% w/w, 90% to 99.5% w/w, or any % or ranges of % therein.

In further embodiments, the compositions comprise biomass, ethylene, abscisic acid, brassinolides, or combination thereof.

In further embodiments, the compositions do not comprise biomass, ethylene, abscisic acid, brassinolides, or any combination thereof.

In further embodiments, the plants are crops from soil microbe friendly and/or synergistic plant families and subfamilies, including but not limited to the following families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Graminede), Polygonaceae, Rosaceae, Rutaceae, Sapindaceae, Saxifragaceae, Solanaceae, and/or Vitaceae.

Disclosed herein are methods of enhancing cation-uptake or iron-uptake in plants growing in low-cation, low-iron or Iron Deficiency Chlorosis (IDC) prone soil, comprising providing a composition comprising one or more gibberellins and/or one or more auxins and/or salicylic acid and/or one or more jasmonates, and applying an effective amount of the composition to one or more seeds, the low-cation, low-iron or Iron Deficiency Chlorosis (IDC) prone soil, growing media, and/or a cultivated area.

In some embodiments, the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01 to 1 ng/ml, the one or more auxins are at a concentration of 0.001 to 0.5 ng/ml, the salicylic acid is at a concentration of 0.5 to 1 ng/ml, and/or the one or more jasmonates are at a concentration of 0.001 to 0.1 ng/ml; the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentrations or ranges of concentrations therein, and the method further comprises diluting said composition to 25:1 and 150:1 prior to applying the effective amount to the soil.

In some embodiments, the composition is derived from algae, optionally macroalgae and/or microalgae. In some embodiments, the microalgae comprise Chlorella, Spirulina, Nannochloropsis, and/or Scenedesmus, or any combination thereof.

In some embodiments, the effective amount is applied to the low-cation, low-iron or Iron Deficiency Chlorosis (IDC) prone soil, growing media, and/or the cultivated area, and the effective amount is 1 to 5 liters, 1.89 to 3.79 liters (0.5 to 1 gallon), or 1 to 2 gallons per acre.

In further embodiments, the method further comprises diluting said composition to 50:1 to 300:1, 100:1 to 250:1, or 150:1 to 200:1 and performing one or more additional applications of the effective amount the composition.

In some embodiments, the one or more additional applications of the composition are performed at least every 1 to 60 days, 1 to 30 days, 1 to 14 days, 1 to 7 days, or any days or ranges of days therein.

In some embodiments, the effective amount is applied to the low-cation, low-iron or Iron Deficiency Chlorosis (IDC) prone soil, growing media, and/or the cultivated area, and the applying comprises injection, in-furrow, drip irrigation, center-pivot, surface broadcast, broadcast incorporated, band application, fertigation, chemigation, foliar application, sidedress, topdress, seed placement and/or seed treatment, or any combination thereof.

In some embodiments, the one or more gibberellins comprise any one of GA1, GA3, GA4, GA5, GA6, and/or GA7 or any combination thereof.

In some embodiments, the one or more auxins comprise any one of Me-IAA, IAA-ALA, IAA-ASP, IBA, or any combination thereof.

In some embodiments, the one or more jasmonates comprise jasmonic acid.

In some embodiments, the composition further comprises cultured and enhanced sterilized water, optionally at 80% to 99% w/w, 90% to 99.5% w/w, or any % or ranges of % therein.

In some embodiments, the composition does not comprise biomass, ethylene, abscisic acid, and/or brassinolides or any combination thereof.

In some embodiments, the composition is applied to soil containing any of the following plant families Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioidee, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineue), Polygonaceae, Rosaceae, Rutaceae, Sapindaceae, Saxifragaceae, Solanaceae, and/or Vitaceae.

Disclosed herein are methods of enhancing cation-uptake or iron-uptake in plants growing in low-cation, low-iron or Iron Deficiency Chlorosis (IDC) prone soil, comprising providing an algae culture and air to an algae culture broth composition producing system, the system comprising: a sterilizer; an automatic carbon dioxide supply device to promote photosynthesis; an at least partially sealed or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and a high-efficiency harvesting device using hollow fiber membranes; and harvesting the composition by separating the composition from biomass of the algae culture using the harvesting device, optionally substantially dehydrating the composition for storage and transport, and diluting it prior to use, and applying an effective amount of the composition to one or more seeds, the low-cation, low-iron or Iron Deficiency Chlorosis (IDC) prone soil, growing media, and/or a cultivated area.

In further embodiments, the microalgae culture comprises Chlorella, Spirulina, Nannochloropsis, and/or Scenedesmus or any combination thereof.

In further embodiments, the composition comprises one or more gibberellins and/or one or more auxins and/or salicylic acid and/or one or more jasmonates.

In some embodiments, the one or more gibberellins are at a concentration of 0.01 to 1 ng/ml, the one or more auxins are at a concentration of 0.001 to 1 ng/ml, the salicylic acid is at a concentration of 0.1 to 1 ng/ml, and/or the one or more jasmonates are at a concentration of 0.001 to 1 ng/ml; the one or more gibberellins are at a concentration of 0.01 to 1 ng/ml, the one or more auxins are at a concentration of 0.001 to 0.5 ng/ml, the salicylic acid is at a concentration of 0.5 to 1 ng/ml, and/or the one or more jasmonates are at a concentration of 0.001 to 0.1 ng/ml; the one or more gibberellins are at a concentration of 0.01 to 0.8 ng/ml, the one or more auxins are at a concentration of 0.001 to 0.4 ng/ml, the salicylic acid is at a concentration of 0.5 to 0.75 ng/ml, and/or the one or more jasmonates are at a concentration of 0.001 to 0.004 ng/ml; or any concentration or ranges of concentrations therein, further comprising diluting said composition to 25:1 to 150:1 or 100:1 prior to applying the effective amount.

In some embodiments, the effective amount is applied to the low-cation, low-iron or Iron Deficiency Chlorosis (IDC) prone soil, growing media, and/or the cultivated area and the effective amount is 1 to 5 liters, 1.89 to 3.79 liters (0.5 to 1 gallon), 1 to 2 gallons, or any volume or ranges therein per acre.

In some embodiments, the method further comprises diluting said composition to 50:1 to 300:1 and performing one or more additional applications of the effective amount the composition.

In some embodiments, the one or more additional applications of the composition are performed at least every 1 to 60 days, 1 to 50 days, 1 to 30 days, 1 to 14 days, 1 to 7 days, or any days or ranges of days therein.

In some embodiments, the effective amount is applied to the low-cation, low-iron or Iron Deficiency Chlorosis (IDC) prone soil, growing media, and/or the cultivated area and the applying comprises injection, in-furrow, drip irrigation, center-pivot, surface broadcast, broadcast incorporated, band application, fertigation, chemigation, foliar application, sidedress, topdress, seed placement and/or seed treatment, or any combination thereof.

In some embodiments, the one or more gibberellins comprise any one of GA1, GA3, GA4, GA5, GA6, and/or GA7 or any combination thereof.

In some embodiments, the one or more auxins comprise any one of Me-IAA, IAA-ALA, IAA-ASP, IBA, or any combination thereof.

In some embodiments, the one or more jasmonates comprise jasmonic acid.

In some embodiments, the composition further comprises cultured and enhanced sterilized water, optionally at 80% to 99% w/w, 90% to 99.5% w/w, or any % or ranges of % therein.

In some embodiments, the composition does not comprise biomass, ethylene, abscisic acid, and/or brassinolides or any combination thereof.

In some embodiments, the composition is applied to soil containing any of the following plant families Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.

Reference will now be made in detail to embodiments, examples of which are illustrated in the accompanying drawings, wherein like reference numerals refer to like elements throughout. In this regard, the present embodiments can have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### EXAMPLES

### Example 1. Preparation of an Algal Composition

An algae culture broth producing system is described in WO2018/009575. The system is further modified, described as follows.

In WO2018/009575, the device for culture broth sterilization comprises a micro air bubble generator; an air compression and pressure equalization device for the injection of carbon dioxide and oxygen from the atmosphere into the culture broth; an air chilling device to maintain suitable culture broth temperature in response to a water temperature being higher than a predetermined maximum temperature; an automatic carbon dioxide supply device to promote photosynthesis; a sealed vertical photobioreactor configured to contain a culture medium inoculated with an algae, the vertical photobioreactor being configured to allow light into the culture medium, block out pollutants and increase dissolved carbon dioxide and oxygen concentration; a high-efficiency harvesting device using hollow fiber membranes (HFMs); and a hot air drying device using the waste heat generated by air compression. The modified device does not contain an air chilling device; closed photobioreactors; and hot air-drying device.

An air chilling device to maintain suitable culture broth temperature in response to a water temperature being higher than a predetermined maximum temperature is replaced with an air dryer without a temperature control device.

A sealed vertical photobioreactor configured to contain a culture medium inoculated with an algae, the vertical photobioreactor being configured to allow light into the culture medium, block out pollutants and increase dissolved carbon dioxide and oxygen concentration is replaced with a system that is not closed: each and every tube has a hole that exposes it to the elements.

A high-efficiency harvesting device using hollow fiber membranes (HFMs) without a hot air drying device that uses the waste heat generated by air compression is used.

In WO2018/009575, the air pressurizing device consists of a device to compress carbon dioxide and oxygen in the atmosphere to 10 bar, a device (tank) to equalize air pressure, and pipes and a gas backflow preventing device to inject the compressed gases into the culture broth. In the modified system, a simpler pressurization is used, i.e., comprising an air compressor, holding tanks, and a CO2 tank (with valve regulators). In WO2018/009575, the air chilling device is for maintaining an optimum water temperature for each algae species in response to a water temperature being higher than a predetermined optimum temperature. In the modified system, there is an air dryer which slightly lowers the temperature of the algae but it does not control temperature to suit the algae's needs. The automatic carbon dioxide supply device supplies gaseous carbon dioxide into the culture broth using a pH sensor in order to promote photosynthesis in the rapid growth phase.

In WO2018/009575, the algae culture broth producing apparatus is disclosed, wherein the automatic carbon dioxide supply device automatically supplies carbon dioxide when the pH of the culture broth is 7.26 or higher, and automatically cuts off carbon dioxide when the pH of the culture broth is less than 7.26 if the algae is a freshwater algae, and automatically supplies carbon dioxide when the pH of the culture broth is 7.30 or higher, and automatically cuts off carbon dioxide when the pH of the culture broth is less than 7.30 if the algae is a seawater algae. The modified system is set to introduce CO2 at pH 7.8 and close the solenoid valve at pH 7.2

In WO2018/009575, the compressed gas is injected into the bottom of the apparatus to cause gases in air bubble form to rise vertically and cause ripples in the culture broth, keeping algae from attaching to the walls of the photobioreactor, causing the penetration rate of light, an element of photosynthesis, thus increasing the concentrations of the growth factors dissolved carbon dioxide, nitrogen and oxygen to increase and in turn promoting photosynthesis, and oxygen, which is the metabolic product of photosynthesis, is immediately discharged into the atmosphere through a discharge pipe disposed on top of the apparatus. The modified system does not have such a “discharge pipe” installed—there are small holes drilled into the lid of each tube.

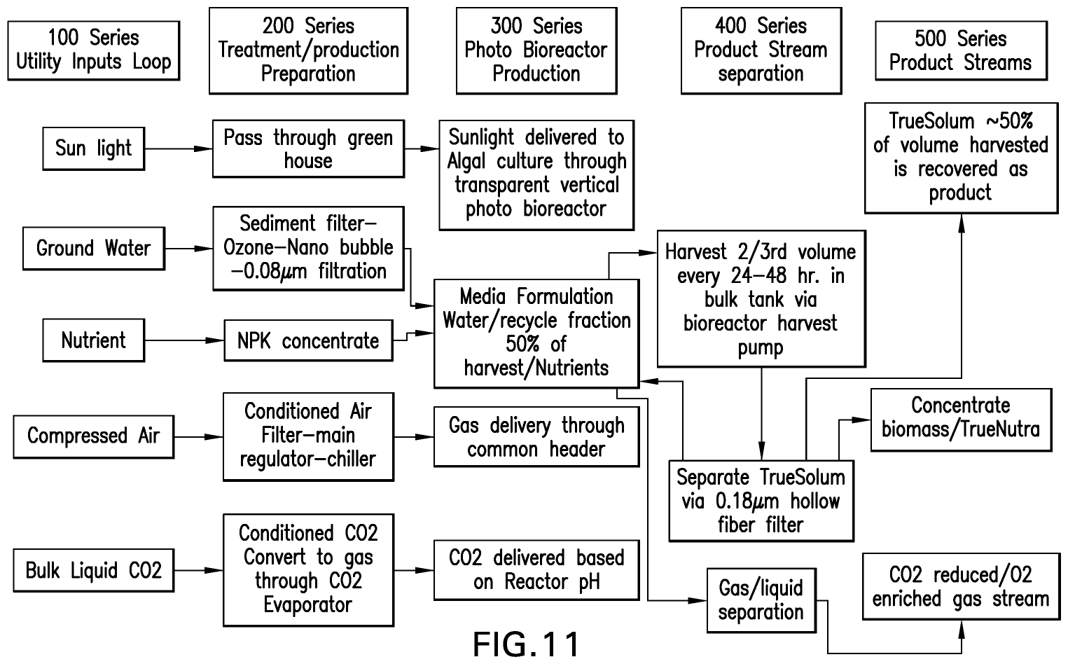

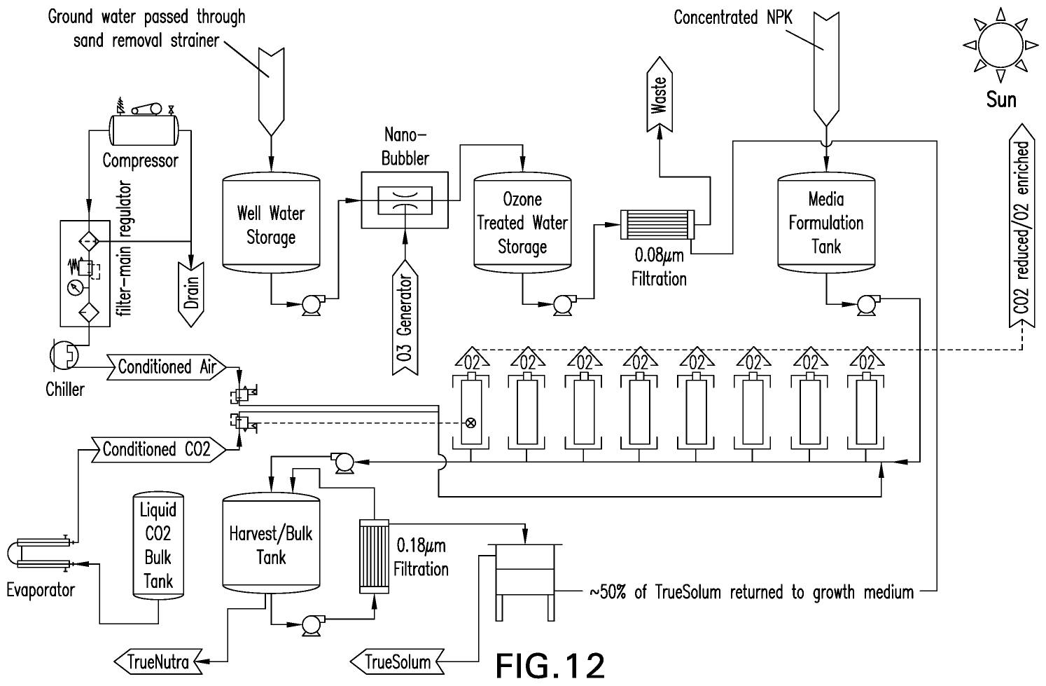

FIG. 11 shows a process flow diagram for producing a composition disclosed herein. FIG. 12 shows an overview of the process for producing a composition disclosed herein.

Using the above-described system, the algal culture and biomass are produced as follows. Water is sanitized using several levels of purification: this redundancy ensures that no potentially harmful organisms remain in the water. A mixture of ozone, high oxygen levels, and microbubbles is dispersed throughout well water and allowed to rest until the gases release from the water itself leaving a sanitized final solution. The final solution is passed through an ultrafiltration hollow fiber membrane to remove any debris or sediment remaining before going into the system.

CO2 is stored in a large liquid CO2 tank outside. It passes through a vaporizer to ensure even release of gas even in extreme temperatures, and then passes through a primary regulator before being regulated again at the divide between the north and south halves of the greenhouse.

Air is generated through rotary screw compressors and stored in large storage tanks to maintain constant air pressure in the system. Before the air is introduced to the PBRs (photobioreactors) it is passed through a dryer, as well as through a final near high-efficiency particulate air (HEPA) level of filtration, and a large regulator to ensure consistent pressure and that too much pressure is not placed on air fittings.

Small levels of liquid fertilizer are mixed into a solution of either water or algae culture medium without biomass product for reintroduction to the PBRs.

The photobioreactors are connected to the system through a main drain/fill tube at the bottom of each unit. Using these we can fill them with sanitized water or a the NPK solution, as well as drain the lots into the main tank for harvest filtration.

Each of the units is connected to both air and CO2. The air is used mostly for even dispersal of the algal culture for optimal sunlight absorption, CO2 dispersal, and general agitation. After they are mixed, the air and the CO2 are regulated a final time at each individual group of PBRs.

Using small pore size hollow fiber membranes, the biomass is removed from the culture broth and separated out as the final product is pumped into a storage tank with little to no algae remaining.

### Example 2. Application of the Composition to Strawberry Plants

A soil genomics study in three separate application trials was performed to assess if microalgae culture medium without biomass product is affected to strawberry plant (Driscoll Odessa, Driscoll Forteleza, and Maverick, Brilliance) soil beneficial microorganisms of bacteria and fungi biodiversity composition and population. For this experiment, the microalgae culture medium without biomass was applied approximately 8 weeks after planting and applied weekly for a total of 6 applications at three geographical locations in the United States (Strawberry Station in Plant City, Florida, Strawberry Ranch in Plant City, Florida, and Eastside Ranch, California) with three replicate soil samples taken at four timepoints (T0, T1, T2, and T3-T0 represents a control where none of the microalgae culture medium without biomass was applied).

In this experiment, the untreated lot was maintained using a standard protocol regarding farming, supplying water, fertilizers, and chemicals. Fifty % of soil samples were taken from within 25 acres. The treated lot was maintained using exactly the same protocol, with the exception of having the microalgae culture medium without biomass applied 6 times weekly at a dosage of 1 gallon/acre, diluted into 250 gallons of water and applied diluted water volume for drip hose irrigation pumping system.

For this experimental study, water and the algal derived microalgae culture medium without biomass supplied to soil of all lots was via drip hose irrigation pumping systems, and fertilizers and chemicals supplied to foliage of all lots was by a chicken wing spray system.

The results of the soil microbiome analysis are shown in Table 1A.

TABLE 1A

| | Percentage Difference with UTC at Same Location | | |
|---|---|---|---|
| | at T₃-T₀ [Q Value] of Stress Adaptation using | | |
| | Delta-Delta Statistical Analysis | | |
| | | | Organic |
| Location of | Siderophore | Potassium (K) | Phosphorous (P) |
| Treatment | Production | Consumption | Assimilation |
| Strawberry Station, | 297.00 [0.21] | 7.87 [0.32] | 6.46 [0.59] |
| Florida (FL) | | | |
| Strawberry Ranch, | 517.88 [0.10] | 0.05 [NA] | 1.46 [0.69] |
| Florida (FL) | | | |

Table 1A shows that lots treated with microalgae culture medium without biomass have increased siderophore production versus the UTC lots with one instance being statistically significant (Location 2). Product treated lots produced environmental stress adaptive abilities resulting in increased production of siderophore microorganisms that produce chelating molecules that increase iron bioavailability and minor nutrient pathway improvements (including, but not limited to, usable iron assimilation) to the plant, particularly in low-cation, low iron, iron deficiency chlorosis (IDC) prone, challenged soils where micronutrients are bound by negatively-charged or cation-chelating herbicides (e.g., Glyphosate (Roundup), Glufosinate (Liberty or Basta), and Atrazine) and insecticides, and calcareous soils, than the untreated lot.

As a result of this superior start, microalgae culture medium without biomass treated lot soils trended consistently and significantly with the following:
Improvement in major nutrient pathways including, but not limited to, increased phosphorous and potassium solubilization);
Improvement in minor nutrient pathways including, but not limited to, increased zinc, iron, and manganese bioavailability); and
Increases in plant hormones, stress-adaptive abilities, and biocontrol agents including, but not limited to, increases in the abundance of most plant growth promoting (PGP) organisms, increased abundance of siderophore producers, and increased nematicide biocontrol microorganisms), and improved disease resistance (including, but not limited to, decreased risk of aspergillus rot, charcoal rot, and hard root rot).

The use of microalgae culture medium without biomass appears to maintain natural soil biodiversity while not directly affecting bacterial or fungi biodiversity (as calculated by the Shannon index) and significantly modified the bacterial and fungal composition of the soil microbiome. By adding microalgae culture medium without biomass, the relative abundance levels of beneficial microorganisms, especially those related to improving iron deficiency issues and resistance to abiotic stress conditions (e.g., suboptimal pH levels, drought, suboptimal salinity levels), including, but not limited to Pseudomonas fluorescens and other beneficial fungi and bacteria, matched or bettered the UTC lots.

Some of the taxa that was represented in this experimental trial that demonstrated the impact of siderophore production and iron assimilation, include, but is not limited to, the following beneficial microrganisms: Aureobasidium_pullulans, Bacillus_sp., Pseudomonas_sp., with some of the resulting fold change percentages versus the control (and with the control variability eliminated so that only the product's effect is reflected and not any external environmental influences (i.e., the impact is generally attributable to the product)) shown in Table 1B.

TABLE 1B

| Location | Timepoint | Microorganism | Fold Change % |
|---|---|---|---|
| Strawberry Station | T1 | Pseudomonas_sp. | 109 |
| Strawberry Station | T1 | Bacillus_sp. | 61 |
| Strawberry Station | T1 | Aureobasidium_pullulans | 20 |
| Strawberry Ranch | T1 | Pseudomonas_sp. | 610 |
| Strawberry Ranch | T1 | Bacillus_sp. | 11 |
| Eastside Ranch | T1 | Pseudomonas_sp. | 24 |
| Strawberry Station | T2 | Pseudomonas_sp. | 940 |
| Strawberry Station | T2 | Bacillus_sp. | 21 |
| Eastside Ranch | T2 | Aureobasidium_pullulans | 3395 |
| Eastside Ranch | T2 | Pseudomonas_sp. | 174 |
| Strawberry Station | T3 | Pseudomonas_sp. | 562 |
| Strawberry Station | T3 | Bacillus_sp. | 7 |
| Strawberry Ranch | T3 | Pseudomonas_sp. | 197 |
| Eastside Ranch | T3 | Aureobasidium_pullulans | 294 |
| Eastside Ranch | T3 | Pseudomonas_sp. | 245 |

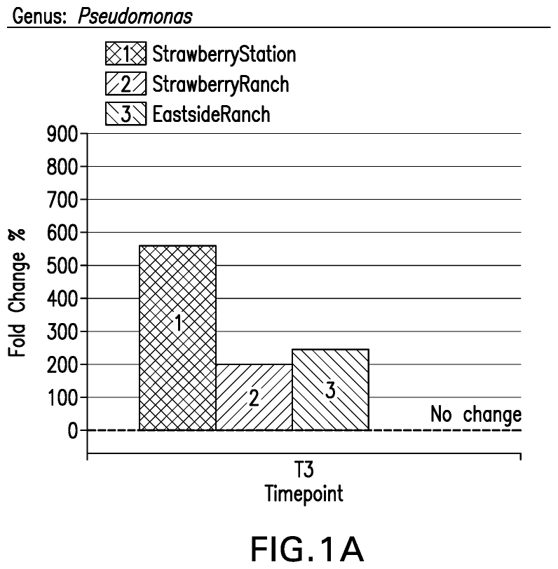

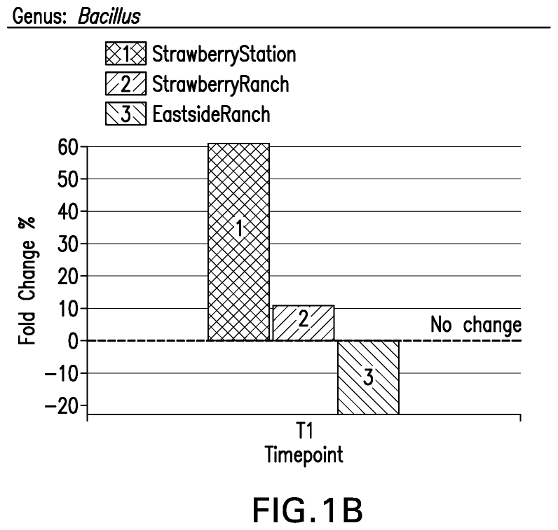

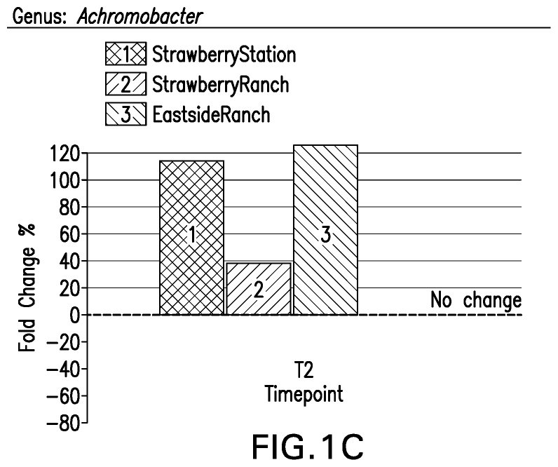

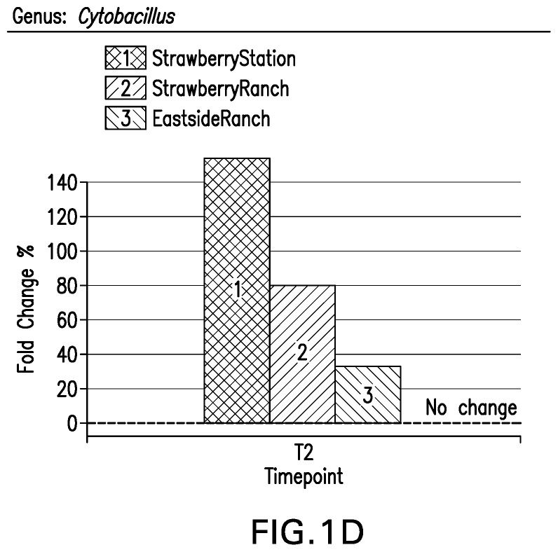

The fold change % in relative abundance versus control is shown in FIG. 1 for Pseudomonas (FIG. 1A), Bacillus (FIG. 1B), Achromobacter (FIG. 1C), and Cytobacillus (FIG. 1D). The data for the relative abundance of Bacillus at the Eastside Ranch is the opposite of the expected result, and contrary to every other data point demonstrated in FIG. 1 for these beneficial species. As such, it is considered that this reduction in relative abundance is artefactual and not due to the treatment with the composition disclosed herein.

Additionally, it is logical that the application of the product improves nutrient uptake and assists in the improvement of natural resistance to abiotic and biotic stresses, as the additional bacteria that were shown to be statistically by in greater relative abundance to the control over time were biocontrol microorganisms, including, but not limited to, Achromobacter_sp (which produces salicylic acid and elicits biocontrol effects, see Forchetti et al., 2010), Trichoderma sp (pathogen suppressing fungi), Bacillus sp, Alkalihalobacillus_sp, and Cytobacillus_sp.

Additionally, it is logical that given herbicide challenged and/or low-iron soils led to stressed plants that have less nutrition to ward off pathogens and have less nutrition that aid in natural defenses (e.g., iron, zinc, manganese), the use of the product was able to positively influence the beneficial microorganisms to release bound key nutrients, improve the efficiency of the uptake of those newly unbound key nutrients, and provide an overall better natural resistance to various environmental stresses.

### Example 3. Treatment of Soybeans with Microalgae Culture Medium without Biomass

An experiment was performed to assess whether microalgae culture medium without biomass impacts soybean early establishment when grown organically.

For this application trial, microalgae culture medium without biomass was applied from planting to harvest (in a period of 7 months from May to November). The soybeans were planted in May, the treatment conditions occurred the next day, and were repeated one month later in June. In September the pods and beans were counted, and weight measurements of mature soybeans were taken. In November, the dry soybean yield measurements were taken.

For this experimental application trial, the untreated lot (5 acres) was maintained using standard practices regarding farming, fertilizer supply and other organic crop inputs. In the microalgae culture medium without biomass treated lot (10 acres), the conditions were identical except for the application of microalgae culture medium without biomass twice within 30 days with a dosage of microalgae culture medium without biomass being 1 gallon/acre, diluted with 100 gallons of water and applied using a vehicle mounted field applicator with a precision dosage flow rate nozzle. In this experiment, the water supplied was from natural precipitation only.

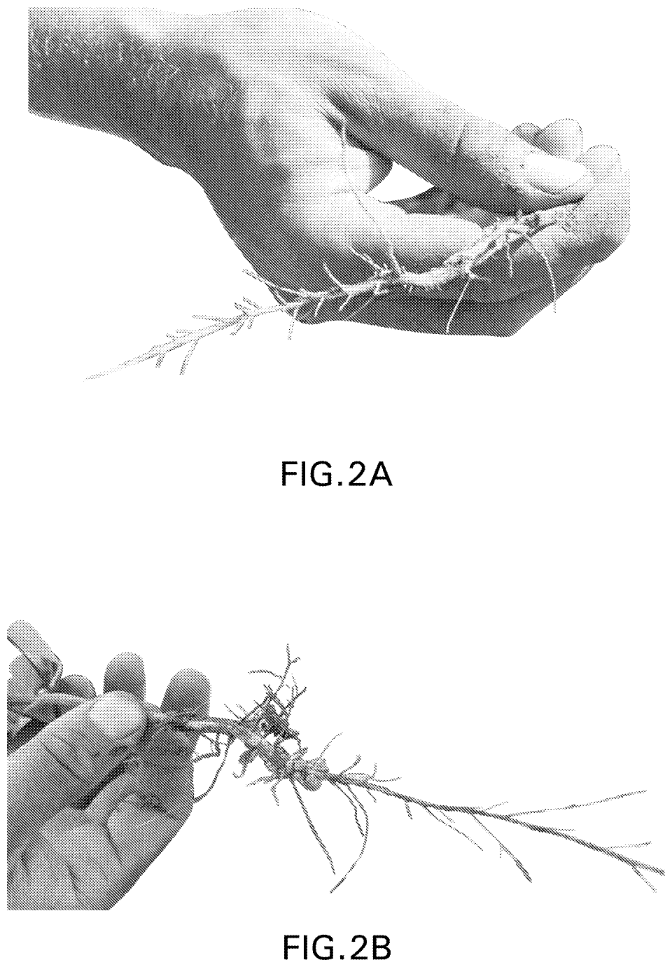

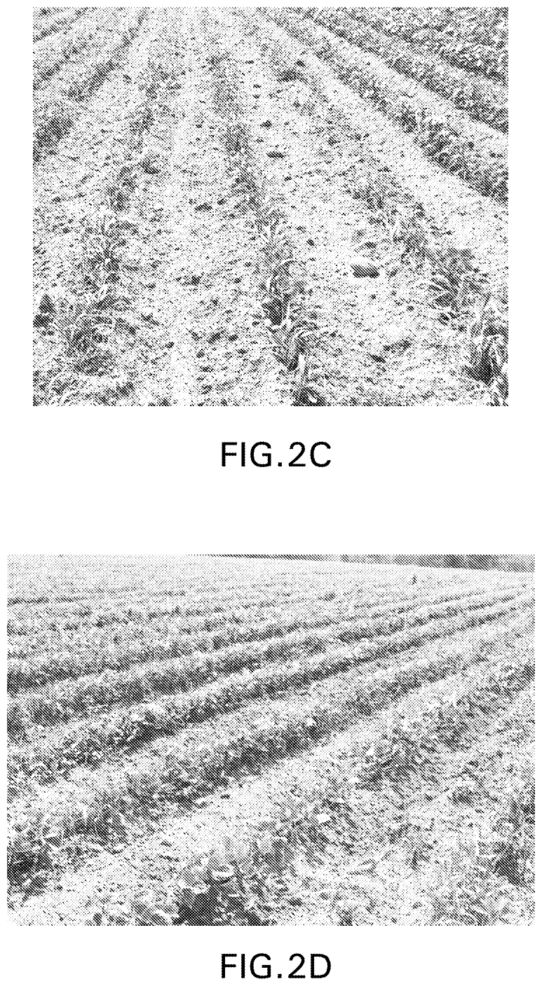

The use of microalgae culture medium without biomass matched or bettered the untreated lots by significantly improving root mass/vigor (FIGS. 2A and 2B), improved nodules count (FIGS. 2A and 2B), accelerated growth to maturity of plant and seed emergence (FIGS. 2C and 2D), improved resistance to severe drought conditions, and fewer bare patches and resultant areas for weed infestation (FIGS. 2C and 2D). Soybeans require a high amount of nitrogen to successfully produce grain. The majority of this is produced in root nodules through a symbiotic relationship with Rhizobium which reside in said nodules. These bacteria convert gaseous atmospheric nitrogen into a form that the plant can utilize. As such, the increased number of nodules after treatment with the product is a good indicator of improved plant health and increased capacity for nitrogen production and uptake. As such treatment with the composition disclosed herein produces an increased number of root nodules, increased branching of the root system, and increased foliar volume in the treated plants compared to the untreated plants. These actions of the composition disclosed herein are thought to be generally applicable to other plant species also.

Additionally, the soil iron (Fe) range was low at 48-62 (within a pH range of 6.3-6.5 in the control lots and low at 45-48 (within pH range of 6.2-6.4) in the product only lots, which suggests there is improved iron uptake efficiency by the plants through the use of the product after only two applications given the following observed improvements:
Improved plant fertility and productivity;
Increased weed suppression; and
Drought resistance observed after a little over 1 month of plant growth activity.

### Example 4. Treatment of Soybeans with Microalgae Culture Medium without Biomass

This trial was performed to assess if microalgae culture medium without biomass is affected to soybean (conventional) early establishment.

For this experimental application trial, product is applied twice during the trial period. The soil was pre-treated in early May, with seeds planted approximately one week afterwards, and the second treatment at the end of June (when the beans are 3/16″ long). In September, the pod count, bean count, and weight measurements of mature soybeans will be taken. In November, the official dry soybean yield measurements will be taken.

In this experiment, the untreated control (8 acres) was maintained using standard protocols relating to farming, fertilizer supply, and chemical supply (including glyphosate). The lot treated with microalgae culture medium without biomass (8 acres) was planted and maintained in exactly the same way as the untreated control, with the exception of the application of microalgae culture medium without biomass twice within 60 days at a dosage of 1 gallon/acre diluted into 25 gallons of water. This was applied using a vehicle mounted 800 gallon tank at a 25 gpa rate applied diluted water volume in furrow. The second treatment was applied at 0.5 gallons/acre, diluted into 25 gallons of water and applied using a vehicle mounted 800 gallon tank at a 25 gpa rate, with the application of the diluted solution being foliar. The water for this experiment was from natural precipitation only, except for the treatments.

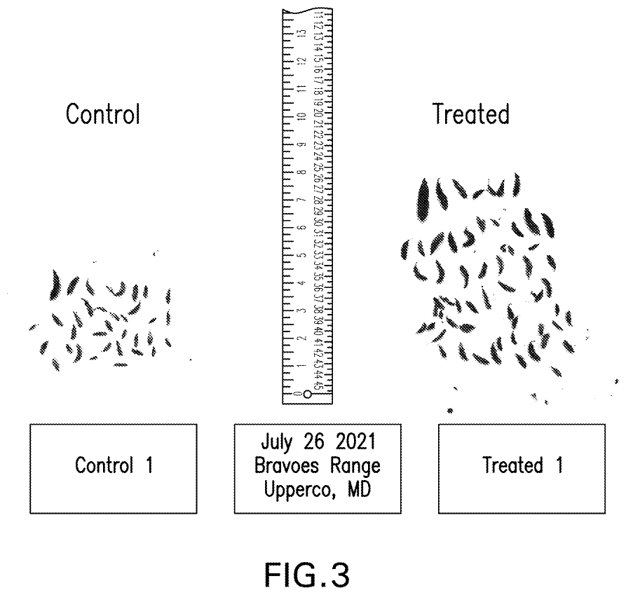

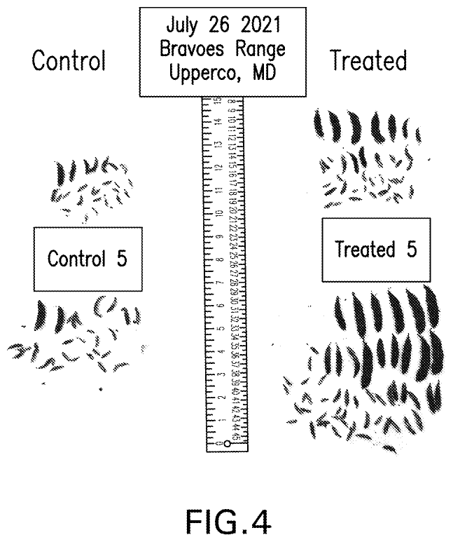

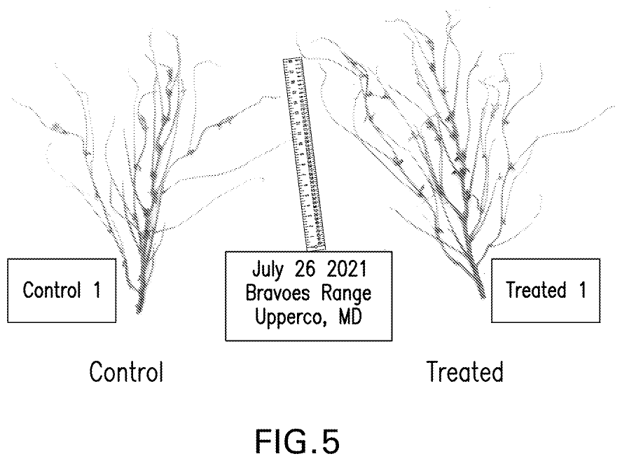

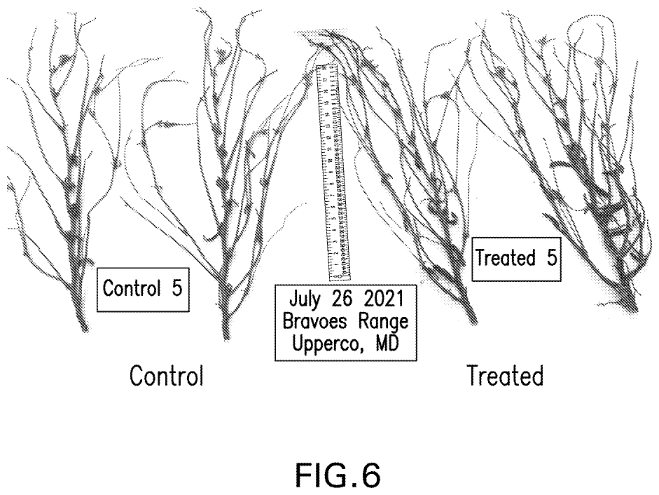

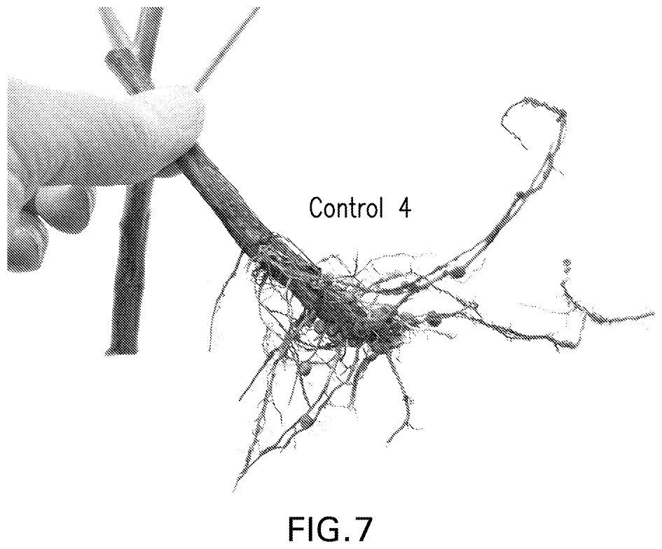

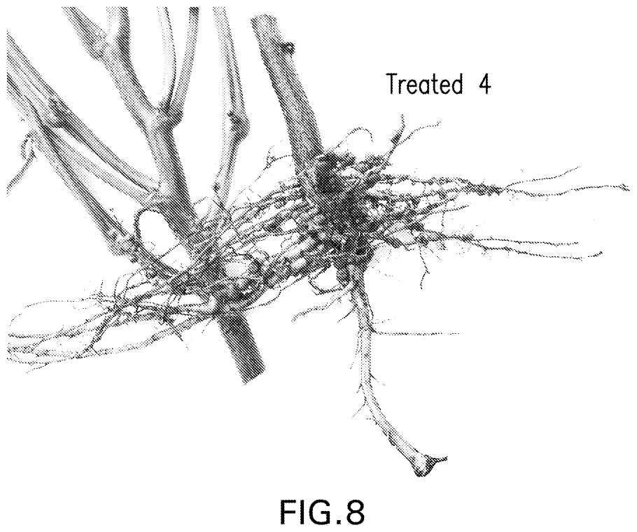

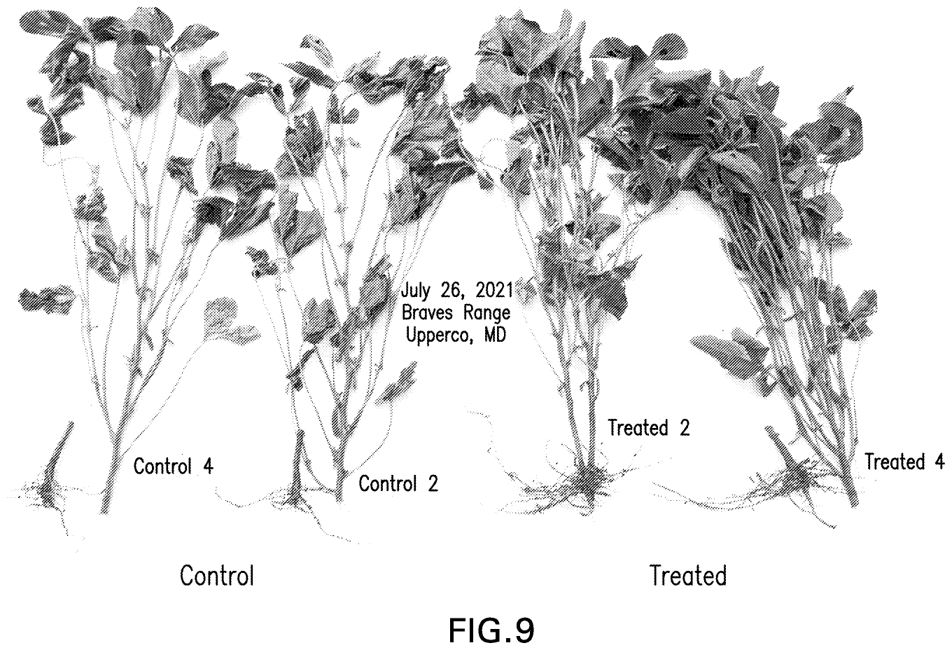

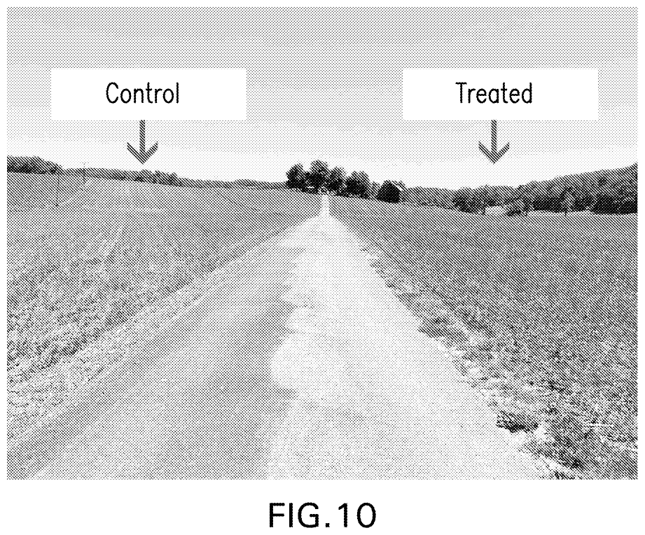

According to field observations, the use of microalgae culture medium without biomass matched or bettered the untreated controls by:
Improving the fertility of the soybeans with increased number of pods produced and increases in the size of pods (FIGS. 3 and 4);
Improving the fertility of the soybeans with increased number of branches (FIGS. 5 and 6);
Improved root nodule count (FIGS. 7 and 8); and
Accelerating growth to maturity of plant, including faster seed emergence, taller plant height, and more leaves (FIGS. 9 and 10).

Results of Haney and Phospholipid Fatty Acid Analysis (PLFA) soil and plant tissue analyses are shown in Table 2.

TABLE 2

| Haney Soil Analysis | Control (ppm) | Treated (ppm) |
|---|---|---|
| Magnesium (Mg) | 56 | 99 |
| Zinc (Zn) | 0.14 | 1.04 |
| Manganese (Mn) | 4.9 | 18.7 |
| Iron (Fe) | 38 | 48 |
| Aluminum (Al) | 69 | 110 |
| Potassium (K) | 103 | 83 |
| Available Phosphorus (P) [lbs/A] | 61.4 | 114.7 |
| Total Phosphorous (P) [ppm] | 26.7 | 49.9 |

| PLFA Soil Analysis | Control (Ratio) | Treated (Ratio) |
|---|---|---|
| Fungi:Bacteria | 0.1243 | 0.1468 |
| Protozoa:Bacteria | 0.0017 | 0.0023 |
| Gram+:Gram− | 0.8814 | 1.6702 |

| | Control (ppm) | | | | | | Treated (ppm) | | | | | |
|---|---|---|---|---|---|---|---|---|---|---|---|---|
| Plant Analysis | Zn | Fe | Mn | Al | % P | % K | Zn | Fe | Mn | Al | % P | % K |
| Tissue (Pod) | 58.30 | 117.40 | 131.01 | 39.40 | 0.61 | 3.22 | 58.57 | 233.06 | 129.10 | 184.87 | 0.72 | 3.69 |
| Tissue (Leaf) | 47.98 | 157.59 | 210.48 | 56.34 | 0.48 | 2.21 | 52.35 | 243.03 | 226.73 | 115.91 | 0.53 | 1.84 |

| Plant Growth | Control | Treated |
|---|---|---|
| Average Plant Height | 26″ | 34″ |

Additionally, the soil iron (Fe) range was high at 70 (with pH of 5.7) in the control lot and high at 84 (with pH of 6.1) in the treated lot. Even though the soil samples are pH low-neutral and there was an abundance of iron in the soil, the use of glyphosate as an herbicide is known to create an environment much like an alkaline soil, thus binding the available iron and other nutrients. The product being applied releases this bound iron and other nutrients, making it available to the plant, which is represented in the plant tissue analyses of both the pod and leaves showing the increased amounts of available iron and other nutrients of the treated over the control.

Additionally, it is plausible and logical that during the final harvesting there will be increased quantity of iron and other nutrients measured in the plant tissues, increased count of pods and beans (final yield), as well as increased root mass, root length, and root nodules of the treated over the control.

### Example 5. Treatment of Strawberry Plants with Microalgae Culture Medium without Biomass

This experiment is performed to assess if microalgae culture medium without biomass product is affected to improve iron (Fe) and other cation transport and assimilation in new strawberry plants.

For this experimental application trial, microalgae culture medium without biomass is applied from planting to mid-harvest (8 weeks) with a 1-week pre-treatment prior to planting. The untreated control lot (25 acres) is maintained using standard farming, water supply, fertilizer supply and chemical supply protocols. The microalgae culture medium without biomass treated lot (25 acres) and Grower's Standard lot (25 acres) are maintained in the same way as the untreated lot, but the Grower's Standard lot is additionally treated with Grower's Standard iron treatment, and the microalgae culture medium without biomass treated lot is additionally supplied 8 times in a 2 month period (4 times per month) with microalgae culture medium without biomass at a dosage of 1 gallon/acre and dilution with 250 gallons of water. The microalgae culture medium without biomass is applied by drip irrigation, and additionally includes a pre-treatment of the soil 10 days prior to planting by a dosage of 1 gallon/acre diluted into 100 gallons of water, again applied by drip irrigation.

For this experimental application trial, water supply to all three groups is through drip hose irrigation pumping systems. Microalgae culture medium without biomass is applied in the middle of the set to allow the system to be at normal operation and allow for rinsing of the hoses. microalgae culture medium without biomass in the drip irrigation system is applied during an approximate 30-minute window followed by several hours of flushing to distribute the product well.

The predicted results of iron (Fe) uptake are shown in Table 3.

TABLE 3

| | | Iron Uptake | Iron Uptake |
|---|---|---|---|
| | | Increase over | Increase over |
| Treatment | Treatment Description | UTC (Soil) | UTC (Tissue) |
| UTC | Control (no micronutrients added) | — | — |
| GS | Grower Standard Iron Treatment | 5-10% | 10-15% |
| TS | Microalgae Culture Medium | 20-30% | 30-50% |
| | without Biomass Product Applied | | |

Table 3 shows that the microalgae culture medium without biomass treated lot increases the amount of available iron in both the soil and within the plant and fruit tissue over both the untreated control, and the lot treated with Grower's Standard iron treatment by 30% and 50%, respectively. The microalgae culture medium without biomass lot is anticipated to enhance the microbial growth and soil health of the field even in soils with high pH and iron deficiency problems.

It is thought that the microalgae culture medium without biomass mitigates the iron issues by providing phytonutrients into the soil microbiome resulting in signaling the proliferation of microbes that produce iron (Fe) chelating siderophores. The improved soil and plant health due to enhancing the soil microbial growth and soil health through the enhanced mobilization and/or solubilization of iron is anticipated to improve the uptake of available iron into the plant and into its resulting tissues and fruit.

### Example 6. Treatment of Almond Plants with Microalgae Culture Medium without

### Biomass

This experiment is performed to assess the effect of microalgae culture medium without biomass product on iron and other cation transport and assimilation in new almond plants.

In this experiment, microalgae culture medium without biomass product is applied from planting to mid-harvest (12 weeks) with a pre-treatment 1 week prior to planting. The untreated control lot (25 acres) is maintained using standard farming, water supply, fertilizer supply, and chemical supply protocols.

The microalgae culture medium without biomass product treated lot (25 acres) and the Grower's Standard lot (25 acres) is maintained the same way, with the following exceptions. The Grower's Standard lot is additionally supplied with Grower's Standard iron treatment, and the microalgae culture medium without biomass product treated lot is additionally supplied with microalgae culture medium without biomass product 12 times in 3 months (4 times a month) at a dosage of 1 gallon/acre diluted into 250 gallons of water, which is applied using drip irrigation. In addition, the soil is pre-treated 10 days prior to planting with 1 gallon/acre of microalgae culture medium without biomass product diluted into 100 gallons of water, again applied by drip irrigation.

The water used in this experiment is supplied through a drip hose irrigation pumping system.

The predicted results of iron (Fe) uptake are shown in Table 4.

TABLE 4

| | | Iron Uptake | Iron Uptake |
|---|---|---|---|
| | | Increase over | Increase over |
| Treatment | Treatment Description | UTC (Soil) | UTC (Tissue) |
| UTC | Control (no micronutrients added) | — | — |
| GS | Grower Standard Iron Treatment | 5-10% | 10%-15% |
| TS | Microalgae Culture Medium | 20-30% | 30%-50% |
| | without Biomass Product Applied | | |

Table 4 shows that the microalgae culture medium without biomass treated lot is anticipated to increase the amount of available iron in both the soil and within the plant and fruit tissue over both the untreated lot, and the lot treated with Grower's Standard iron treatment by 30% and 50%, respectively. The microalgae culture medium without biomass treated lot is anticipated to enhance the microbial growth and soil health of the field even in soils with high pH and iron deficiency problems.

It is logical that the microalgae culture medium without biomass treated lot will mitigate the iron issues by providing phytonutrients into the soil microbiome resulting in signaling the proliferation of microbes that produce iron (Fe) chelating siderophores. The improved soil and plant health due to enhancing the soil microbial growth and soil health through the enhanced mobilization and/or solubilization of iron is anticipated to improve the uptake of available iron into the plant and to its resulting tissues and fruit.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details can be made therein without departing from the spirit and scope of the disclosure as defined by the following claims.

The breadth and scope of the present invention should not be limited by any of the above-described exemplary embodiments but should be defined only in accordance with the following claims and their equivalents.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference.

### REFERENCES

1. Dennis, P. G., Kukulies, T., Forstner, C. et al. The effects of glyphosate, glufosinate, paraquat and paraquat-diquat on soil microbial activity and bacterial, archaeal and nematode diversity. Sci Rep 8, 2119 (2018). https://doi.org/10.1038/s41598-018-20589-6.
2. Forchetti G, Masciarelli O, Izaguirre M J, Alemano S, Alvarez D, Abdala G. Endophytic bacteria improve seedling growth of sunflower under water stress, produce salicylic acid, and inhibit growth of pathogenic fungi. Curr Microbiol. 2010 December; 61 (6): 485-93. doi: 10.1007/s00284-010-9642-1. Epub 2010 Apr. 11. PMID: 20383767.
3. Richard L. Haney, Elizabeth B. Haney, Douglas R. Smith, R. Daren Harmel, Mike J. White, The soil health tool—Theory and initial broad-scale application, Applied Soil Ecology, Volume 125, 2018, Pages 162-168, ISSN 0929-1393, https://doi.org/10.1016/j.apsoil.2017.07.035.
4. Hilal, M. H., Anter, F. & El-Damaty, A. H. A chemical and biological approach towards the definition of calcareous soils. Plant Soil 39, 469-478 (1973). https://doi.org/10.1007/BF00264165
5. Nabeel Imam, Ignacio Belda, Adrian J. Duehl, James R. Doroghazi, Daniel E. Almonacid, Varghese P. Thomas, Alberto Acedo, Soil Microbial Composition and Structure Allow Assessment of Biological Product Effectiveness and Crop Yield Prediction. bioRxiv 2021.02.09.430373; doi: https://doi.org/10.1101/2021.02.09.430373
6. Lindsay, W. L. and Norvell, W. A. (1978), Development of a DTPA Soil Test for Zinc, Iron, Manganese, and Copper. Soil Science Society of America Journal, 42:421-428. https://doi.org/10.2136/sssaj1978.03615995004200030009x
7. Malo, D. 2013. Identifying potential iron chlorosis soils for soybeans production. In Clay, D. E., C. G. Carlson, S. A. Clay, L. Wagner, D. Deneke, and C. Hay (eds). iGrow Soybean: Best Management Practices for Soybean Production. South Dakota State University, SDSU Extension, Brookings, SD.
8. Mertens, M., Höss, S., Neumann, G. et al. Glyphosate, a chelating agent—relevant for ecological risk assessment?. Environ Sci Pollut Res 25, 5298-5317 (2018). https://doi.org/10.1007/s11356-017-1080-1
9. R. H. Loeppert (1986) Reactions of iron and carbonates in calcareous soils, Journal of Plant Nutrition, 9:3-7, 195-214, DOI: 10.1080/01904168609363437
10. Sakal, R., et al. “Determination of Threshold Value of Iron in Soils and Plants for the Response of Rice and Lentil to Iron Application in Calcareous Soil.” Plant and Soil, vol. 82, no. 1, 1984, pp. 141-148.
11. Zobiole, L., Kremer, R., Oliveira, R., Jr and Constantin, J. (2011), Glyphosate affects micro-organisms in rhizospheres of glyphosate-resistant soybeans. Journal of Applied Microbiology, 110:118-127. https://doi.org/10.1111/j.1365-2672.2010.04864.

What is claimed is:

1. A method of increasing cation uptake in a plant growing in soil treated with an herbicide or pesticide, comprising:
(a) applying an effective amount of an herbicide or pesticide to the plant and/or soil; and
(b) within 10 days after the applying in (a), applying to the plant and/or soil an effective amount of a composition comprising one or more gibberellins, one or more auxins, salicylic acid, and/or one or more jasmonates.

2. The method of claim 1, wherein the applying in (a) and (b) occur concurrently or on the same day.

3. The method of claim 1, wherein the applying in (b) occurs 1 to 9 days after the applying in (a).

4. The method of claim 1, wherein the applying in (b) occurs 1 to 5 days after the applying in (a).

5. The method of any one of claims 1-4, further comprising applying the composition every 1 to 60 days after the applying in (b).

6. The method of any one of claims 1-5, wherein prior to an optional dilution of the applying in (b):
the one or more gibberellins are at a concentration of 0.01 to 1 ng/ml;
the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml.

7. The method of any one of claims 1-6, wherein the composition is derived from algae, macroalgae, and/or microalgae.

8. The method of claim 7, wherein the microalgae comprise Chlorella, Spirulina, Nannochloropsis, and/or Scenedesmus or combinations thereof.

9. The method of any one of claims 1-8, wherein the one or more gibberellins comprise GA1, GA3, GA4, GA5, GA6, GA7, or combinations thereof.

10. The method of any one of claims 1-9, wherein the one or more auxins comprise Me-IAA, IAA-ALA, IAA-ASP, IBA, or combinations thereof.

11. The method of any one of claims 1-10, wherein the one or more jasmonates comprise jasmonic acid.

12. The method of any one of claims 1-11, wherein the composition further comprises cultured and enhanced sterilized water, optionally at 80% to 99.5% w/w.

13. The method of any one of claims 1-12, wherein the composition comprises biomass, ethylene, abscisic acid, brassinolides, or combinations thereof.

14. The method of any one of claims 1-12, wherein the composition does not comprise biomass, ethylene, abscisic acid, brassinolides, or combinations thereof.

15. The method of any one of claims 1-14, wherein the plant is from any of the following families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.

16. A method of enhancing cation-uptake or iron-uptake in plants growing in low-cation soil, low-iron soil, or Iron Deficient Chlorosis (IDC)-prone soil, comprising applying an effective amount of a composition comprising one or more gibberellins, one or more auxins, salicylic acid, and/or one or more jasmonates to one or more seeds, the soil, growing media, and/or a cultivated area.

17. The method of claim 16, wherein a herbicide or pesticide is applied to the one or more seeds, the soil, growing media, and/or a cultivated area concurrently, on the same day, or within 10 days before the composition is applied.

18. The method of claim 17, wherein
the one or more gibberellins are at a concentration of 0.01 to 1 ng/ml;
the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml; and
further comprising diluting the composition to 25:1 and 150:1 prior to applying the effective amount.

19. The method of any one of claims 16-1-8, wherein the composition is derived from algae, macroalgae, and/or microalgae.

20. The method of claim 19, wherein the microalgae comprise Chlorella, Spirulina, Nannochloropsis and/or Scenedesmus or any combination thereof.

21. The method of any one of claims 16-20, wherein the effective amount is applied to the soil, growing media, and/or the cultivated area, and wherein the effective amount is 1 to 5 liters per acre.

22. The method of any one of claims 16-21, further comprising diluting the composition 50:1 and 300:1 and performing one or more additional applications of the effective amount of the composition.

23. The method of claim 22, further comprising additionally applying the composition every 1 to 60 days.

24. The method of any one of claims 16-23, the effective amount is applied to the soil, growing media, and/or the cultivated area, and wherein the applying comprises injection, in-furrow, drip irrigation, center-pivot, surface broadcast, broadcast incorporated, band application, fertigation, chemigation, foliar application, sidedress, topdress, seed placement, seed treatment, or combinations thereof.

25. The method of any one of claims 16-24, wherein the one or more gibberellins comprise GA1, GA3, GA4, GA5, GA6, GA7. or combinations thereof.

26. The method of any one of claims 16-25, wherein the one or more auxins comprise Me-IAA, IAA-ALA, IAA-ASP, IBA, or combinations thereof.

27. The method of any one of claims 16-26, wherein the one or more jasmonates comprise jasmonic acid.

28. The method of any one of claims 16-27, wherein the composition further comprises cultured and enhanced sterilized water, optionally at 80% to 99.5% w/w

29. The method of any one of claims 16-28, wherein the composition comprises biomass, ethylene, abscisic acid, brassinolides or any combinations thereof.

30. The method of any one of claims 16-28, wherein the composition does not comprise biomass, ethylene, abscisic acid, brassinolides or combinations thereof.

31. The method of any one of claims 16-30, wherein the composition is applied to soil containing any of the following plant families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.

32. A method of producing a composition that increases cation uptake in a plant growing in soil and growing the plant, comprising providing an algae culture and air to an algae culture broth composition producing system, the system comprising:
a sterilizer;
an automatic carbon dioxide supply device to promote photosynthesis;
an at least partially or fully sealed vertical photobioreactor configured to contain a culture medium inoculated with an algae, the vertical photobioreactor being configured to allow light into the culture medium, at least partially block out pollutants and increase dissolved carbon dioxide and oxygen concentration; and
a high-efficiency harvesting device using hollow fiber membranes;
harvesting the composition; and
applying the composition to the plant and/or the soil.

33. The method of claim 32, wherein the composition is harvested by separating the composition from biomass of the algae culture using a harvesting device, optionally dehydrating the composition for storage and transport, and optionally diluting it prior to use.

34. The method of claim 32 or 33, wherein the algae culture comprises Chlorella, Spirulina, Nannochloropsis, and/or Scenedesmus, or any combination thereof.

35. The method of any one of claim 32-34, wherein the composition comprises one or more gibberellins, one or more auxins, salicylic acid, and/or one or more jasmonates.

36. The method of any one of claims 32-35, wherein the one or more gibberellins comprise GA1, GA3, GA4, GA5, GA6, GA7 or combinations thereof.

37. The method of any one of claims 32-36, wherein the one or more auxins comprise Me-IAA, IAA-ALA, IAA-ASP, IBA, or combinations thereof.

38. The method of any one of claims 32-37, wherein the one or more jasmonates comprise jasmonic acid.

39. The method of any one of claims 32-38, wherein the composition comprises biomass, ethylene, abscisic acid, brassinolides or combinations thereof.

40. The method of any one of claims 32-39, wherein the composition does not comprise biomass, ethylene, abscisic acid, brassinolides or combinations thereof.

41. A mixture comprising an herbicide or pesticide and a composition comprising:
one or more gibberellins, one or more auxins, salicylic acid, and/or one or more jasmonates.

42. The mixture of claim 41, wherein prior to an optional dilution, the composition comprises:
the one or more gibberellins are at a concentration of 0.01 to 1 ng/ml;
the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml.

43. The use of a composition to enhance the iron-uptake in plants growing in low-cation, low-iron, or Iron Deficient Chlorosis (IDC)-prone soils, the composition comprising:
one or more gibberellins;
one or more auxins;
salicylic acid; and/or
one or more jasmonates.

44. The use of claim 43, wherein, prior to an optional dilution at the point of application:
the one or more gibberellins are at a concentration of 0.01 to 1 ng/ml;
the one or more auxins are at a concentration of 0.001 to 1 ng/ml;
the salicylic acid is at a concentration of 0.1 to 1 ng/ml; and/or
the one or more jasmonates are at a concentration of 0.001 to 1 ng/ml.

45. The use of claim 43 or 44, wherein the composition is derived from algae, optionally macroalgae and/or microalgae.

46. The use of any one of claims 43-45, wherein the algae comprise Chlorella, Spirulina, Nannochloropsis, and/or Scenedesmus or any combination thereof.

47. The use of any one of claims 43-46, wherein the one or more gibberellins comprise GA1, GA3, GA4, GA5, GA6, GA7 or combinations thereof.

48. The use of any one of claims 43-47, wherein the one or more auxins comprise Me-IAA, IAA-ALA, IAA-ASP, IBA, or combinations thereof.

49. The use of any one of claims 43-48, wherein the one or more jasmonates comprise jasmonic acid.

50. The use of any one of claims 43-49, wherein the composition further comprises cultured and enhanced sterilized water, optionally at 80% to 99.5% w/w.

51. The use of any one of claims 43-50, wherein the composition comprise biomass, ethylene, abscisic acid, brassinolides or combinations thereof.

52. The use of any one of claims 43-50, wherein the composition does not comprise biomass, ethylene, abscisic acid, brassinolides or combinations thereof.

53. The use of any one of claims 43-52, wherein the plants are crops from any of the following families: Actinidiaceae, Adoxaceae, Allaceae, Amaranthaceae, Anacardiaceae, Apiaceae (Umbelliferae), Arecaceae, Asteraceae, Bromeliaceae, Cactaceae, Caesalpinioideae, Cannabaceae, Capparaceae, Chenopodiaceae, Cucurbitaceae, Ericaceae, Fabaceae, Lamiaceae (Labiatae) Lauraceae, Liliaceae, Lythraceae, Moraceae, Musaceae, Myrtaceae, Oleaceae, Oxalidaceae, Papilionaceae, Passifloraceae, Poaceae (Gramineae), Polygonaceae, Rosaceae, Rutaceae, Sapindaceae, Saxifragaceae, Solanaceae, and/or Vitaceae.
